(19) United States
(12) Patent Application Publication
(10) Pub. No.: US 2002/0169123 A1
(43) Pub. Date: Nov. 14, 2002

(54) Regulating apoptosis in TRAIL-resistant cancer cells, while protecting normal, non-cancerous cells

(75) Inventors: Wafik S. El-Deiry, Bryn Mawr, PA (US); Kunhong Kim, (US)

Correspondence Address: DILWORTH PAXSON LLP, 3200 MELLON BANK CENTER, 1735 MARKET STREET, PHILADELPHIA, PA, 19103, US

(73) Assignee: THE TRUSTEES OF THE UNIVERSITY OF PENNSYLVANIA, Philadelphia, PA (US)

(21) Appl. No.: 10/085,801

(22) Filed: Feb. 27, 2002

Related U.S. Application Data
(60) Provisional application No. 60/271,674, filed on Feb. 27, 2001.

(51) Int. Cl. A61K 38/17

(52) U.S. Cl. 514/12

(57) ABSTRACT

Provided are compositions and methods for controlling, modulating or regulating extrinsically-induced apoptosis in a population of cells, comprising treating the cell population with a synergistically combined composition comprising an amount of TRAIL in conjunction with an amount of at least one reagent acting on mitochondrial pathways of the cells, which in combination is sufficient to induce cellular apoptosis, such that the apoptosis-inducing effect of the combination is greater than that of TRAIL alone, or the at least one reagent alone, or the additive individual apoptotic effects of TRAIL and the at least one reagent. However, TRAIL-sensitive normal cells are protected from the extrinsically induced apoptosis by treatment with a specific caspase inhibitor, such as a caspase 9 inhibitor. Consequently, in accordance with the present invention, TRAIL-resistant cancer cells are treated and killed with an apoptosis-inducing amount of the TRAIL combination, but the normal cells are protected or rescued from apoptosis by treatment with the specific caspase inhibitor.

### RELATED APPLICATION

This application claims the benefit of U.S. Provisional Application No. 60/271,674, filed Jan. 27, 2001, which filing date is claimed herein, and the content of which is herein incorporated by reference.

### FIELD OF THE INVENTION

The present invention provides novel insights into the cellular control and regulation of the extrinsic pathway of apoptosis in a population of cells, particularly cancer cells, but not normal, non-cancerous cells. Also provided are compositions and therapeutic treatments useful in the regulation of apoptosis.

### BACKGROUND OF THE INVENTION

Apoptosis, or programmed cell death (PCD), and the cell death pathways have become the subject of intense study in recent years because of recognized links to many physiological and pathological processes, including embryogenesis, differentiation, development of the immune system, autoimmunity, elimination of virus-infected cells, the maintenance of tissue homeostasis, and cancer (Thompson, Science 267:1456-1462 (1995)). Apoptosis, is fundamentally distinct from degenerative death or necrosis, wherein cell death occurs as the result of injury, infection or damaging changes in the cellular environment. Apoptosis is an active, rather than a passive process, resulting in gene-directed cellular self-destruction and suicide, which in some instances, serves a biological homeostatic function. For a general review of apoptosis, see Tomei et al., Apoptosis: The Molecular Basis of Cell Death (1991) Cold Spring Harbor Press, N.Y.; Tomei et al., Apoptosis II: The Molecular Basis of Apoptosis in Disease (1994) Cold Spring Harbor Press, N.Y.; and Duvall et al., Immun. Today 7(4):115-119 (1986). Diseases and conditions in which apoptosis has been implicated fall into two categories, those in which there is increased cell survival (i.e., apoptosis is reduced) and those in which there is excessive cell death (i.e., apoptosis is increased). Morphologically, apoptosis is characterized by the rapid condensation of the cell with preservation of membranes. Several biochemical changes occur in the cell in synchrony as the chromatin is compacted. Nuclear DNA is cleaved at the linker regions between nucleosomes to produce fragments, which are easily separated by agarose gel electrophoresis resulting in a characteristic ladder.

Pioneering studies by numerous investigators to identify a complement of genes related to cell death have revealed an entire family of proteases in mammals, comprising at least 14 different members of the caspase family (cystein-containing aspartate-specific protease) (Wyllie, J. Pathol. 153:313-316 (1987); Thornberry et al., Science 281:1312-1316 (1998); Cohen, Biochem. J. 326:1-16 (1997)) and signals that involve multiple organelles including the mitochondria, the endoplasmic reticulum and endosomes that bud off of the cell membrane. Caspases are synthesized as inactive proenzymes.

The caspases involved in apoptosis have been divided into two groups based on their structure and function. The long prodomain “initiator” caspases include caspases 8, 9, 2 and 10. These initiator caspases contain an amino-terminal domain that becomes cleaved during activation. For example, in the intrinsic cell death pathway the mitochondria is involved, and caspase 9 activation is controlled positively by mitochondrial cytochrome c release, but it is negatively controlled by the IAPs (inhibitors of apoptosis proteins). The short prodomain “executioner” caspases include caspases 3, 6, and 7. These executioner caspases are activated by the initiator caspases. Once activated, the executioner caspases cleave hundreds-thousands of cellular proteins resulting in the phenotype of cell death.

The TNF-related apoptosis inducing ligand (TRAIL or Apo2L) is a potent inducer of death of cancer cells, but not normal cells, when it binds to either of two pro-apoptotic TRAIL receptors, TRAIL R1 (DR4) (Pan et al., Science 276:111-113 (1997)) or TRAIL R2 (KILLER/DR5) (Wu et al., Nat. Genet. 17:141-143 (1997); Pan et al., Science 277:815-818 (1997); Walczak et al., EMBO J. 16:5386-5397 (1997); Screaton et al., Curr. Biol. 7:693-696 (1997); Wu et al., Adv. Exp. Med. Biol. 465:143-151 (2000)). Activation of the pro-apoptotic death receptors by TRAIL engagement induces the formation of death inducing signaling complex (DISC) which consists of receptor, FADD as an adaptor (Kischkel et al., Immunity 12:611-620 (2000); Kuang et al., J. Biol. Chem. 275:25065-25068 (2000)), and caspase 8 as an initiator caspase. Once DISC is formed, caspase 8 is auto-processed and activated by induced proximity (Muzio et al., J. Biol. Chem. 273:2926-2930 (1998); Medema et al., EMBO J. 16:2794-2804 (1997)).

In FAS/FASL cell death tranducing receptor-mediated apoptosis, two types of apoptotic signaling pathways exist, i.e., type I and type II (Scaffidi et al., EMBO J. 17:1675-1687 (1998); Scaffidi et al., J. Biol. Chem. 274:22532-22538 (1999)). In type I cells, the activated caspase 8 can directly activate downstream caspase 3, without the involvement of the mitochondrial pathway. On the other hand, in type II cells, there is little caspase 8 recruitment into DISC. Although not enough to activate caspase 3, the caspase 8 is enough to activate the mitochondrial pathway in type II apoptosis. In the presence of z-LEHD-FMK (fluoro-methyl ketone modification of peptide LEHD (SEQID NO:1)), a specific caspase 9 inhibitor, caspase 3 is activated in some cancer cells, but not in the other cancer cells (Ozoren et al., Cancer Res. 60:6259-6265 (2000)), suggesting that similar types of pathways may be present in TRAIL-mediated apoptosis.

The Bcl-2 family of proteins represent adaptors needed for the activation of the caspases, and exemplify an apoptosis-target activation-assisting polypeptide that could be modulated to effect an indirect change in the activation of the target compound or polypeptide. The Bcl-2 family includes both apoptosis-promoting (e.g., Bax and Bad) and apoptosis-inhibiting (e.g., Bcl-2 and Bcl-xL) members.

Bid, one of the Bcl-2 homology (BH) 3 domain only protein members, plays an important role in bridging signals from death receptors (extrinsic pathway) to mitochondria (intrinsic pathway). In the extrinsic (TRAIL-mediated) pathway, activated caspase 8 cleaves Bid to form truncated Bid (tBid) (Yamada et al., Biochem. Biophys. Res. Commun. 265:130-133 (1999)), which is then translocated into the mitochondria followed by Bax translocation, BAK oligomerization, cytochrome-c release, and activation of caspase 9 and the other downstream caspases (Luo et al., Cell 94:481-490 (1998); Li et al., Cell 94:491-501 (1998); Korsmeyer et al., Cell Death Differ. 7:1166-1173 (2000); Wei et al., Genes Dev. 14:2060-2071 (2000); Ruffolo et al., Cell Death Differ. 7:1101-1108 (2000); Wei et al., Science 292, 727-730 (2001)).

TRAIL is a promising agent for development as a cancer therapeutic (French et al., Nat. Med. 5:146-147 (1999)) because it appears to specifically kill transformed and cancer cells, whereas most normal cells appear to be resistant to TRAIL (Ashkenazi et al., Science 281:1305-1308 (1998); Walczak et al., Nat. Med. 5:157-163 (1999)). Systemic administration of TRAIL has proven to be safe in mice, and it can kill breast or colon xenografted tumors and prolong survival (Walczak et al., 1999). However, the potential utility and safety of systemic administration of TRAIL has been questioned recently because of results showing sensitivity of human, but not monkey or mouse hepatocytes to recombinant human TRAIL in vitro (Jo et al., Nat. Med. 6:564-567 (2000); Nagata et al., Nat. Med. 6:502-503 (2000)). Although TRAIL can specifically kill many cancer cells, there are still many cancer cells that display TRAIL-resistance (Kim et al., Clin. Cancer Res. 6:335-346 (2000); Zhang et al., Cancer Res. 59:2747-2753 (1999)).

A number of mechanisms have been described that are responsible for TRAIL-resistance. At the receptor level, low or undetectable expression of DR4 (Kim et al., 2000), or over-expression of TRAIL-decoy receptors, such as TRID (DcR1 or TRAIL-R3) (Pan et al., 1997; Sheridan et al., Science 277:818-821 (1997); Degli-Esposti et al., J. Exp. Med. 186:1165-1170 (1997)), or TRUNDD (DcR2, or TRAIL-R4) (Pan et al., FEBS Lett. 424:41-45 (1998); Marsters et al., Curr. Biol. 7:1003-1006 (1997); Degli-Esposti et al., Immunity 7:813-820 (1997); Meng et al., Mol. Ther. 1:130-144 (2000)), can block TRAIL-mediated apoptotic signaling. At the DISC level, loss of caspase 8 expression (Teitz et al., Nat. Med. 6:529-535 (2000); Griffith et al., J Immunol. 161:2833-2840 (1998)) or high expression of c-FLIP (Kim et al., 2000; Zhang et al., 1999; Kataoka et al., J. Immunol. 161:3936-3942 (1998; Scaffidi et al., J. Biol. Chem. 274:1541-1548 (1999)), make cancer cells resistant to TRAIL. At the mitochondria level, high expression of anti-apoptotic Bcl-2 family protein, such as Bcl-2 or Bcl-xL, can block signal propagation if the cells take the type II pathway (Scaffidi et al., 1998; Walczak et al., Cancer Res. 60:3051-3057 (2000)). Loss of expression of Apaf-1, documented in malignant melanoma, could also be a possible mechanism for TRAIL resistance at post mitochondrial level (Soengas et al., Nature 409:207-211 (2001)). In addition, it has been reported that elevated AKT activity confers TRAIL-resistance on LnCap, a prostate cancer cell line, in tissue-type specific manner (Nesterov et al., J. Biol. Chem. 276:10767-10774 (2001)).

Esophageal cancer constitutes about 1.5% of newly diagnosed invasive malignancies and 2% of all cancer related deaths in the United States annually (Yang et al., Clin. Cancer Res. 6:1579-1589 (2000)). Thus, although TRAIL has proven to be therapeutically effective for the selective killing of cancer cells in a patient, a need has remained in the art until the present invention to selectively kill TRAIL-resistant cancer cells, without killing the cells in the patient's surrounding normal tissue.

### SUMMARY OF THE INVENTION

The present invention meets the need in the art and identifies and provides compositions and methods for controlling, modulating or regulating extrinsically-induced apoptosis in a population of cells comprising treating the cell population with a synergistically combined composition, comprising an amount of TRAIL in conjunction with an amount of at least one reagent acting on mitochondrial pathways of the cells, which in combination is sufficient to induce cellular apoptosis, and wherein the apoptotic effect of TRAIL alone or the at least one reagent alone, or the additive individual apoptotic effects of TRAIL and the at least one reagent, fail to achieve the apoptotic effect on the cell population of the synergistic combination (the TRAIL combination). However, otherwise TRAIL-sensitive normal cells are protected from the extrinsically induced apoptosis by treatment with a specific caspase inhibitor, such as a caspase 9 inhibitor, preferably z-LEHD-FMX. Consequently, in accordance with the present invention, TRAIL-resistant cancer cells are treated and killed with an apoptosis-inducing amount of the TRAIL combination, but the normal cells are protected or rescued from apoptosis by treatment with the specific caspase inhibitor.

To better understand TRAIL-mediated apoptosis, human esophageal tissue, including eight esophageal cancer cell lines and one normal primary esophageal epithelial cell were examined. As a result, normal primary esophageal epithelial cells (EPC2) were found to be sensitive, even at low concentrations of TRAIL. But in the presence of a specific caspase inhibitor, such as a caspase 9 inhibitor, for which z-LEHD-FMK was the exemplified species, the normal cells were completely protected from apoptotic cell killing, as verified in human hepatocytes. In addition, pre-treatment of z-LEHD-FMK into mixed cultures of TRAIL-sensitive cancer cells+EPC2 cells, preferentially protected the primary esophageal epithelial cells from TRAIL-mediated apoptosis (TRAIL-resistance), while the TRAIL-sensitive cancer cells remained sensitive to TRAIL or to the TRAIL combination.

By comparison cycloheximide (CHX) pre-treatment was found to sensitize all of the esophageal cancer cells irrespective of the presence or absence of c-FLIP expression, suggesting the existence of short-half lived inhibitory molecule(s) besides c-FLIP acting at the level of caspase 8 activation.

It is an object of the invention in accordance with a preferred embodiment of the foregoing methods to treat the cell population with the components of the synergistic combination by first administering TRAIL, followed by the at least one reagent acting on the mitochondrial pathway. In particular, the population of cells being treated comprises cancer cells. More particularly, the preferred treatment of the cancer cells comprises treating TRAIL-resistant cancer cells. In fact, treating the population of cells with the synergistic combination further comprises causing at least a portion of the TRAIL resistant cancer cells to become sensitive to apoptosis extrinsically induced by the synergistic combination.

It is a further object of the invention, when the population of cells further comprises normal, non-cancerous cells, sensitive to apoptosis extrinsically induced by TRAIL or the synergistic TRAIL combination, to treat the cells with an amount of a specific caspase inhibitor sufficient to induce resistance to the synergistic combination. As a result, apoptosis extrinsically induced by the synergistic combination is prevented, inhibited, or blocked, and resistance to the synergistic TRAIL combination is effected in the normal cells, but the sensitive cancer cells are not so protected. In fact, the apoptosis protection is also afforded to “rescue” cells already undergoing apoptosis. The preferred specific caspase inhibitor for the present compositions and methods is a caspase 9 inhibitor; more preferably it is z-LEHD-FMX.

It is yet another object of the invention to provide to the TRAIL combination, at least one reagent acting on mitochondrial pathways of the cells. In particular, the reagent is a recognized chemotherapeutic composition, or it is produced in vivo as a result of a chemotherapeutic treatment. The reagent is thus a chemotherapeutic composition selected from the group consisting of doxorubicin (adriamycin), cisplatin, carboplatin, etoposide, cyclophosphamide, 5FU, CPT11 (irinotecan), taxol or derivatives thereof, vincrisitine and vinerolbine, or it is produced by a chemotherapeutic treatment, such as radiation.

In the presence of a caspase specific inhibitor, specifically a caspase 9 inhibitor, preferably z-LEHD-FMK, relatively low Bcl-xL-expressing cancer cells were synergistically killed, although they had been previously TRAIL-resistant. However, normal esophageal epithelial cells (EPC2 cells) and other cells, including cancer cells, that had high basal levels of Bcl-xL expression were almost completely protected from TRAIL-induced cell apoptosis.

It is a further object of the invention to provide the compositions of matter, specifically the synergistic TRAIL combination used in the foregoing methods.

It is also an object of the invention to provide a therapeutic method of treating a cancer patient by administering to the patient having a population of cancer cells an apoptosis inducing amount of a synergistically combined composition comprising an amount of TRAIL in conjunction with an amount of at least one reagent acting on mitochondrial pathways of cancer cells, which in combination is sufficient to induce cellular apoptosis, wherein the effect of TRAIL alone or the at least one reagent alone, or the additive individual effects of TRAIL and the at least one reagent, fail to achieve the cellular apoptosis effect of the synergistic combination in a patient. The effect on the cells of the patient is either systemic or localized to a selected population of cells.

It is a further object of the invention, when the patient being treated further has a population of normal, non-cancerous cells, sensitive to apoptosis extrinsically induced by TRAIL or the synergistic TRAIL combination, to treat the cells with an amount of a specific caspase inhibitor sufficient to induce resistance to the synergistic combination. As a result, apoptosis extrinsically induced by the synergistic combination is prevented, inhibited, or blocked, and resistance to the synergistic TRAIL combination is effected in the normal cells, but the sensitive cancer cells are not so protected. In fact, the apoptosis protection is also afforded to “rescue” cells already undergoing apoptosis. The preferred specific caspase inhibitor for the present compositions and methods is a caspase 9 inhibitor; more preferably it is z-LEHD-FMX.

It is an additional object of the invention to provide constructs for administering TRAIL or the TRAIL combination or for providing the controlled expression of same. For instance a construct is provided comprising an adenovirus-controlled expression of an isoleucine-zipper fused to at least one extra-cellular portion of TRAIL.

Additional objects, advantages and novel features of the invention will be set forth in part in the description, examples and figures which follow, and in part will become apparent to those skilled in the art on examination of the following, or may be learned by practice of the invention.

### DESCRIPTION OF THE DRAWINGS

The foregoing summary, as well as the following detailed description of the invention, will be better understood when read in conjunction with the appended drawings. For the purpose of illustrating the invention, there are shown in the drawings, certain embodiment(s), which are presently preferred. It should be understood, however, that the invention is not limited to the precise arrangements and instrumentalities shown.

FIG. 1 shows TRAIL cytotoxicity on eight different esophageal cancer cell lines, as labeled, in the presence or absence of cycloheximide (CHX), as compared with an untreated control. Experiments were done duplicate (mean value±SD).

FIG. 2 depicts an immunostained western blot showing the relative expression level (TRAIL-sensitivity) of the molecules involved in TRAIL-mediated apoptosis. Cell lysates of the eight esophageal cancer cell lines from FIG. 1, plus normal epithelial esophageal cells (EPC2) were prepared, separated by SDS-PAGE electrophoresis, and then immunostained with the antibodies indicated on the left side of FIG. 2. The cell lines are identified across the top of FIG. 2.

FIGS. 3A and 3B depict immunostained western blots showing the sensitization of TRAIL-resistant HCE4 cells to TRAIL by CHX pre-treatment.

FIG. 3A depicts the western blot of a DISC IP analysis to determine the recruitment of caspase 8 into DISC with (+), or without (−), the pre-treatment of CHX in HCE4, TE9 and TE12 cells. Using equal amounts of total cell lysates, the expression levels of c-FLIP were compared with an actin standard. “Stimulation+” means that TRAIL cross-linked with anti-6× His antibody was added to the cells before lysis. “Stimulation−” means the addition of TRAIL to the cells after lysis.

FIG. 3B depicts a 4-hour time-course analysis of the activation of caspase 8, Bid, caspase 9, caspase 3, and the cleavage of PARP by TRAIL, with (+), or without (−), pre-treatment of CHX in TRAIL-resistant HCE4 cells. Lysates were prepared at time points every 0.5 hours after addition of TRAIL. Immunostaining was performed with the indicated antibodies.

FIGS. 4A-4D depict the preferential cytoprotective effect of z-LEHD-FMK on TRAIL-mediated apoptosis in esophageal cells.

FIG. 4A graphically compares apoptotic cell death in an active caspase 3 assay, among HFK, EPC2, and TE2 cells exposed to increasing concentrations of TRAIL over a 4-hour time course. Experiments were done in duplicate (mean value±SD).

FIG. 4B graphically depicts the results of an active-caspase 3 assay to determine the effect on apoptosis resulting from treatment either with TRAIL alone, or with TRAIL plus adriamycin, in the presence, or absence, of z-LEHD-FMK on EPC2 normal esophageal epithelial cells over a 4-hour time course.

FIG. 4C graphically depicts the results of an active-caspase 3 assay to determine the effect of z-LEHD-FMK on TRAIL-sensitive esophageal cancer cells, matching the conditions shown in FIG. 4A.

FIG. 4D depicts the results of an active-caspase 3 assay to confirm the preferential protection of z-LEHD-FMK on normal esophageal epithelial cells in TRAIL-mediated apoptosis, but not on TRAIL-sensitive cells. The cells were grown as a mixed culture containing EPC2-GFP cells (marked with green fluorescent protein) and TRAIL-sensitive TE2 cells, and treated with TRAIL over a 4-hour time course in the presence, or absence, of z-LEHD-FMK. The two cell populations were distinguished by the presence (EPC2-GFP), or absence (TE2), of GFP. A bar graph appears in FIG. 4E, at the right of the western blot to quantitatively display the results shown in FIG. 4D. Experiments were done duplicate (mean value±SD).

FIGS. 5A-5D depict the synergistic cell killing that occurs in TRAIL-resistant esophageal cancer cells when treated with a combination of adriamycin and TRAIL.

FIG. 5A is a western blot depicting the results of an active-caspase 3 assay for apoptosis in TRAIL-resistant HCE4, TE3, and TE5 cells after treatment with TRAIL, adriamycin (Adria), and a combined treatment of adriamycin (Adria) and TRAIL (Adria+TRAIL/no inhibitor), and matched cells treated with z-LEHD-FMK followed by the combined treatment of adriamycin+TRAIL (Adria+TRAIL/C9I).

FIG. 5B depicts the change in mitochondrial membrane potential (Δψₘ) following adriamycin treatment, as shown by a change in DiOC₆ fluorescence when analyzed by flow cytometry.

FIG. 5C is a western blot depicting the translocation of Bax from the cytoplasm to the mitochondria as a result of treatment with adriamycin.

FIG. 5D compares the cell lysates used in FIG. 2 to shown the basal expression levels of Bcl-xL in EPC2, HCE4, TE9, TE3, and TE5 cells.

FIG. 5E is a western blot showing the effect of suppressing the endogenous Bcl-xL in TE5 cells using either the control morpholino (“Control” or “C” in inset box), or anti-sense Bcl-xL morpholino (“AS Bcl-xL” or “AS” in the inset box) oligonucleotides treatment of the cells. Insets in the figures show the expression levels of Bcl-xL in the cells following the corresponding oligonucleotide treatments.

FIGS. 6A-6F depict the expression, secretion and apoptosis-inducing activity of a constructed TRAIL-expressing adenovirus.

FIG. 6A schematically diagrams the constructs used to prepare Ad-GFP and Ad-ILZ-TR.

FIG. 6B is a western blot depicting the expression and secretion of TRAIL-resistant TE5 cells following infection with Ad-GFP or Ad-ILZ-TR. The arrowhead in FIG. 6B indicates unprocessed, signal peptide-containing TRAIL, and the arrow in FIG. 6B represents the fully processed forms of TRAIL.

FIG. 6C depicts the oligomerization of secreted TRAIL.

FIG. 6D depicts the apoptosis inducing activities of expressed TRAIL. TRAIL-sensitive SW480 cells were infected with Ad-GFP (FIG. 6D, a and c), or Ad-ILZ-TR (FIG. 6D, b and d), and the cells were observed post-infection under a bright field (FIG. 6D, a and b) or a fluorescent (FIG. 6D, c and d) microscope (×400).

FIG. 6E is a western blot of the lysates obtained from the same SW480 cells as those used in FIG. 6D showing the effect on apoptosis resulting from the addition of z-VAD-FMK post-infection. Caspase 3 and PARP were cleaved in the cells infected with Ad-ILZ-TR (FIG. 6E, lane 2), but no cleavage was observed (FIG. 6E, lane 4) in the SW480 cells protected by z-VAD-FMK.

FIG. 6F is a western blot showing the effect of the viruses (Ad-ILZ-TR derived TRAIL) on TRAIL-sensitive TE2 cells, TRAIL-resistant TE5 cells, and normal esophageal epithelial EPC2 cells. The TE2 cells were apoptotic (FIG. 6F, TE2), but the TE5 cells were resistant to infection (FIG. 6F, TE5). The EPC2 cells were shown by bright field and fluorescent microscopy (×400) to be undergoing apoptosis (FIG. 6F; b and e; lanes 2 and 8), but in the presence of z-LEHD-FMK, the normal cells were completely protected from cell death (FIG. 6F; c and f; lanes 3 and 9).

### DETAILED DESCRIPTION OF PREFERRED EMBODIMENTS

The present invention identifies and provides compositions and methods for modulating, regulating or controlling extrinsically-induced apoptosis in a suitable cell or a population of suitable cells, by introducing into the cell or cells an effective amount of protein or polypeptide or combination of proteins or polypeptides (collectively referred to herein as an “apoptosis regulating polypeptide,” specifically TRAIL or TRAIL+reagent(s) that act on the mitochondrial pathway of the cell), or a specific caspase inhibitor, as compared with a comparable control cell or cell population. A “specific” caspase inibitor is selectively effective on only one caspase within the caspase cascade of activation steps leading to apoptosis, e.g., the caspase 9 inhibitor is a specific caspase inhibitor affecting only the activation of caspade 9. However, the cascade is typically stopped at whatever point the chain of activation events is broken. Therefore, while the inhibition is specific, the general effect applies to all caspases activated in the cascade after the inhibited member, and apoptosis is blocked, unless the cell is not subject to such effect.

Even though TRAIL is a potent inducer of apoptosis (“cell death”) in cancer cells, there are still many cancers that are not killed by TRAIL (“TRAIL-resistant cancer cells”). Many successful approaches have been proposed to overcome the resistance, such as the combination of TRAIL with other reagents, namely DNA damaging agents (Kim et al, 2000; Nagane et al., Cancer Res. 60:847-853 (2000)), ionizing radiation (Chinnaiyan et al., A., Proc. Natl. Acad. Sci. USA 97:1754-1759 (2000)), or wild type p53 expressing virus (Kim et al., 2001).

In the present invention, however, the effect of TRAIL and a novel combination of TRAIL and a reagent that acts on the mitochondrial pathway of the cell (e.g., but not limited to, adriamycin) and its method of use is demonstrated in at least 8 human esophageal cancer cell lines and in one primary normal esophageal epithelial cell type (EPC2). Three of the cancer cell lines were sensitive to TRAIL, the remainder were TRAIL-resistant esophageal cancer cell lines.

A “cell population” is a group of the same or similar cells in a particular environment, wherein the cells react in substantially the same way to stimuli. Rapidly proliferating cancer cells represent a population of cells, as do populations of cells found in a selected organ or organelle of the body, such as the retina of the eye. Cells are considered to be “TRAIL-sensitive” when more than 30% of the cell population expresses the active form of caspase 3. Cells are considered to be “TRAIL-resistant” when more than approximately 50% of the cell population survives exposure to TRAIL.

“Apoptosis” is recognized in the art as an active process of gene-directed cellular self-destruction. The process is also referred to as “programmed cell death,” and in the present invention as “cell killing.” In a preferred embodiment, apoptosis is extrinsically induced, enhanced or activated, wherein particularly in cancer cells, the introduced TRAIL protein or polypeptide, or a combination of TRAIL+reagent(s) that acts on the mitochondrial pathway of the cell (hereinafter referred to as the “TRAIL combination”), or the nucleic acid molecule(s) encoding same, has sufficient apoptosis-inducing biological activity to activate or enhance apoptosis. In one preferred embodiment, the introduced apoptosis-regulating polypeptide or apoptosis-regulating gene has the ability to modulate, regulate or control activation of at least one member in the caspase signaling cascade, effecting activation of apoptosis of a population of cells.

An “apoptosis-inducing amount of apoptotic polypeptide” is meant that amount of a polypeptide or combination thereof, which is effective or sufficient to induce, enhance or activate a percentage of cells undergoing apoptosis (the effective amount) in a given population of cells capable of undergoing apoptosis, such as cancer cells, relative to a population of untreated control cells of the same type. Preferably, the induced apoptosis is at least 10%, more preferably at least 20%, even more preferably at least 50%, most preferably 70% or even more preferably 90% or more of the treated cells relative to a population of untreated control cells of the same type. Thus, the combined level of killed cells and cells undergoing apoptosis (that absence intervention will be soon killed) in a selected cell population upon treatment with an apoptosis-inducing amount of apoptotic polypeptide (also the effective amount) is at least 2-fold greater, preferably at least 3-fold greater, more preferably at least 4-fold greater, and most preferably at least 5-fold or more greater than a comparable population of untreated control cells of the same type. An “apoptosis-inducing amount,” “apoptosis-enhancing amount” or “apoptosis activating amount” would have the same meaning.

Apoptosis-sensitivity varies by cell type. For example, normal esophageal epithelial cells are susceptible to TRAIL-mediated apoptosis, even at low concentrations (5, 10, 25 or 50 ng/ml) of TRAIL. By comparison, normal human foreskin keratinocytes are not susceptible to apoptosis at those concentrations. Consequently certain types of normal cells need greater levels of protection from apoptosis than other cell types, especially when they are proximate to the targeted cancer cells being treated with apoptosis-inducing amounts of an apoptosis-regulating polypeptide.

In accordance with preferred embodiments of the present invention, synergistic cell killing was observed both in TRAIL-resistant cancer cells, and in the normal EPC2 cells, when the cells were treated with the TRAIL combination. As used in the present invention “synergy” or “synergistic cell killing” refers to cell death or killing of cells by, for example, a combination of two agents, which together achieve a greater effect (more cells killed) than would be expected by simply adding together the cell killing achieved by either of the two agents alone. Suitable reagents that act on the mitochondrial pathway of the cell, and which are suitable for combination with TRAIL in accordance with the preferred embodiments of the present invention, include, but are not limited to, all chemotherapeutics, for example, doxorubicin (adriamycin), cisplatin, carboplatin, etoposide, cyclophosphamide, 5FU, CPT11 (irinotecan), taxol or derivatives, vincrisitine and vinerolbine, to name but a few. Moreover, the reagent acting on mitochondrial pathways of the cells may be produced in vivo as a result of a chemotherapeutic treatment, rather than administered per se. For example, at least one such reagent is produced as a result of chemotherapeutic radiation.

The reagent (acting on the mitochondrial cell pathway) when combined with TRAIL in the TRAIL combination, was found to be effective in amounts lower than the standard chemotherapeutic dose for a given agent. This is because the synergistic extrinsic apoptosis-inducing effect of TRAIL+the reagent in combination in a population of cells achieves an apoptotic effect on the cells which is greater than the effect of TRAIL alone or the effect of the reagent alone, or greater than the additive individual effects of TRAIL and the reagent. Thus, less of the reagent is needed to cause the apoptosis effect in the combination (the effective amount) than it would otherwise be expected if the reagent were used to act on the mitochondrial pathway of the cells without TRAIL. Effective amounts of the reagent vary depending on body surface area, tumor type and whether other agents are included in the combination, as well as in some cases, renal function or any history of toxicity, including myelosuppression.

Moreover, although the description is directed to a reagent, it is also intended to include combinations of same. Thus, “reagent” actually refers to at least one reagent, but two or more reagents in combination or collectively produced in vivo are encompassed within the meaning of the invention.

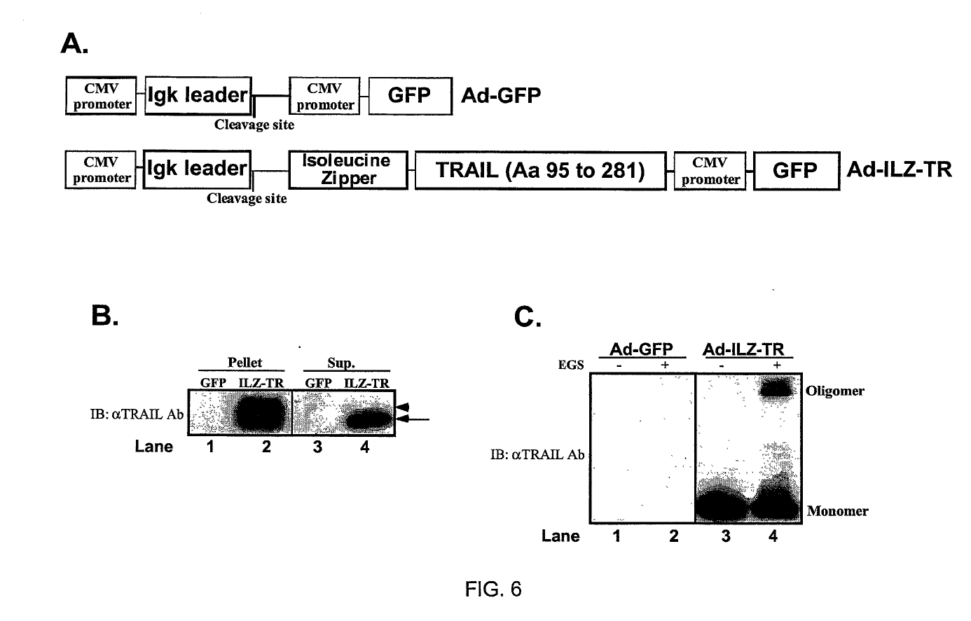

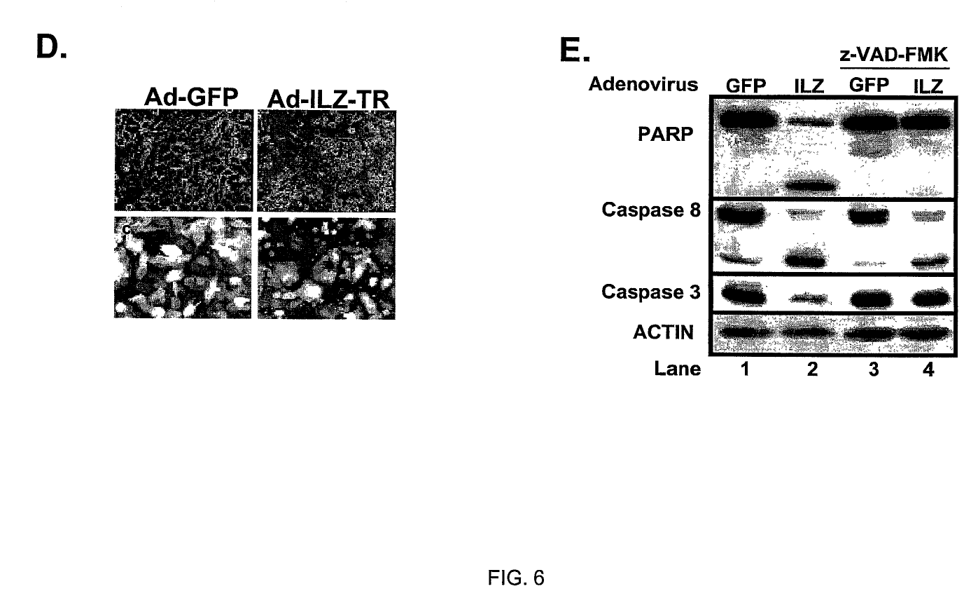

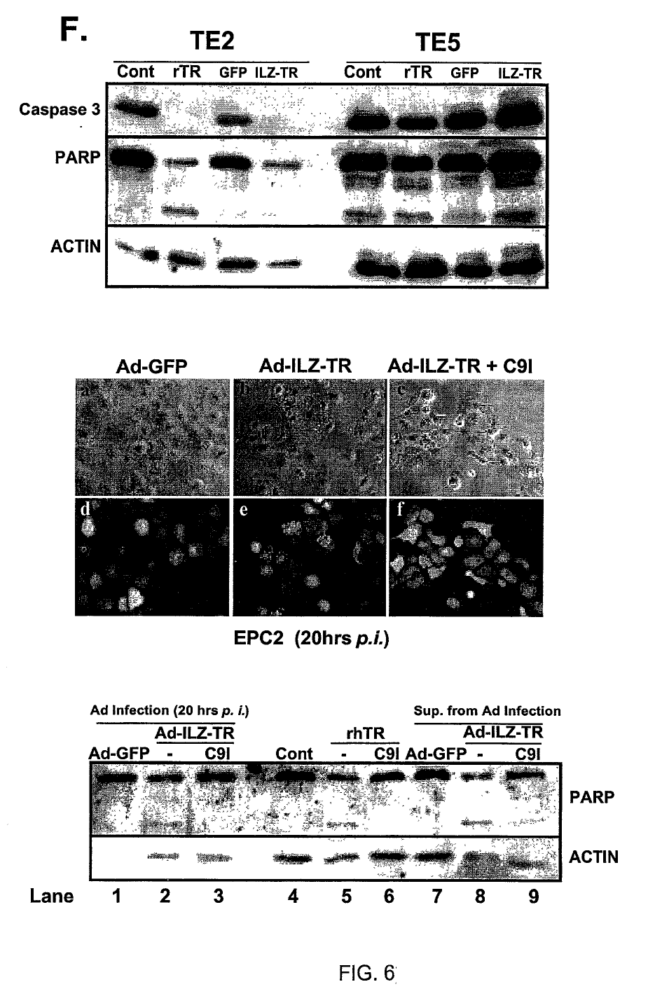

In addition, in preferred embodiments of the invention, methods are provided for preventing apoptosis in certain types of cells and for rescuing normal cells from apoptosis, particularly as shown in the Examples that follow, wherein epithelial cells are rescued from the apoptotic synergistic killing effect of the TRAIL combination. Further provided are methods for protecting such normal cells from TRAIL-induced apoptosis when TRAIL is combined as taught. Although EPC2 cells were found to be TRAIL-sensitive to apoptosis, in a preferred embodiment of the invention, treatment of the normal cells (e.g. EPC2 cells) with a caspase 9 inhibitor (e.g., but not limited to, z-LEHD-FMK), completely protected the cells from apoptotic cell death induced both TRAIL and the TRAIL combination. In fact, in FIG. 6 in is shown that the addition of z-LEHD-FMK effectively “rescued” virtually all of those EPC2 cells that were already undergoing TRAIL+adriamycin-induced apoptosis, and it prevented apoptosis in those cells in which the apoptosis process had not yet begun. By “rescued” refers to ‘being allowed to survive’ and, in the case of proliferating cells, being allowed to proliferate (if the cell type normally proliferates).

Irreversible caspase inhibitors suitable for use in the present invention to protect or rescue selected cells from the apoptosis-inducing effects of TRAIL or the TRAIL combination, include, but are not limited to caspase 2 inhibitor and z-LEHD-FMK (caspase 9 inhibitor). Neither z-IETD-FMK (caspase 8 inhibitor) or z-VAD-FMK (Pan caspase inhibitor) are useful for the purposes of the present invention since they protect all cells, including cancer cells, from the killing effect of TRAIL. In a preferred embodiment of the invention the apoptosis-inducing effect is prevented or the cells are rescued by z-LEHD-FMK. Even small amounts of the caspase 9 inhibitor were found to be effective.

As demonstrated in the Examples which follow, the “rescued” cells were analyzed (see Example 6; FIG. 6F; b and e; lanes 2 and 8) following exposure to TRAIL+adriamycin, and confirmed to be in the process of apoptosis. But when z-LEHD-FMK was subsequently added, the normal EPC2 cells were protected from cell death (“rescued;” see FIG. 6F; c and f; lanes 3 and 9). Moreover, if the EPC2 cells were treated with z-LEHD-FMK before exposure to the TRAIL+adriamycin combination, the normal epithelial cells were completely protected from the apoptotic killing effect that the combination would have otherwise had on the cells.

In accordance with the methods of the present invention, treatment with the synergistic combination further comprises exposing the cell population first to TRAIL, followed by the at least one reagent acting on the mitochondrial pathway. Caspase 9 inhibitor protects normal cells from TRAIL, and so the cancer cells are killed in a few hours while the normal cells are protected. This is followed within 12 hours by chemotherapy, administered in the absence of the caspase 9 inhibitor, in order to obtain maximum effects of the chemotherapy in killing residual cancer cells that were not killed by TRAIL alone.

There are a number of features are routinely use in combination to identify apoptosis in a population of cells (apoptotic cells), including Annexin V positivity (early marker), loss of mitochondrial membrane potential, the presence of active caspase 3 (intermediate marker), PARP (polyADP-ribose-ribose polymerase) cleavage, and the uptake of propidium iodide (late marker). In addition, morphological signs are routinely scored, including cell shrinkage, nuclear condensation and chromatin fragmentation, which is visualized, for example, by DAPI staining.

Nevertheless, in the three cancer cell lines tested that were sensitive to TRAIL, the cancer cells were still killed despite being exposed to z-LEHD-FMK. When the effect of the TRIAL combination (TRAIL+adriamycin) was tested on TRAIL-resistant esophageal cancer cells and EPC2 cells in the presence of z-LEHD-FMK, a large percentage of the previously resistant cancer cells were killed. However, while the EPC2 were completely protected from synergistic cell killing, a small number of TRAIL-resistant cancer cells were also protected by the addition of z-LEHD-FMK.

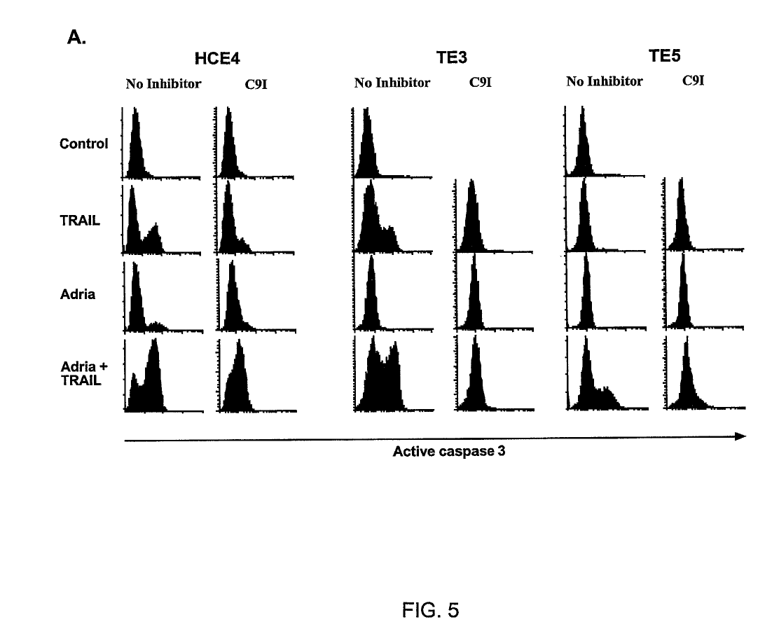

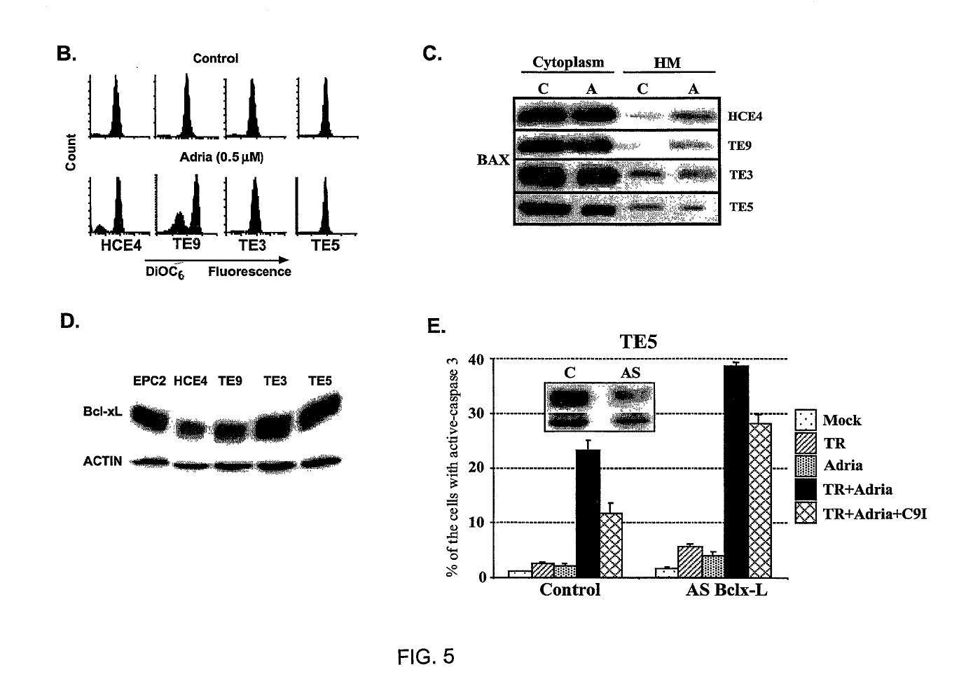

This finding was correlated with the basal expression level of the Bcl-2 family of adaptor proteins needed for the activation of the caspase cascade, specifically with the level of apoptosis-inhibiting protein Bcl-xL. It was determined that the cancer cells (e.g., HCE4 and TE9 cells) that were killed, even in the presence of z-LEHD-FMK showed low basal expression of Bcl-xL (see Example 5; FIG. 5D), whereas high Bcl-xL expressing cancer cells were not killed. It was further observed that a drop of mitochondrial membrane potential (Δψₘ) and Bax translocation following adriamycin treatment (as shown by a change in DiOC₆ fluorescence when analyzed by flow cytometry) were also apparent only in those high-Bcl-xL expressing cells (FIGS. 5B, 5C and 5E). However, the presence of the Bcl-xL inhibitor did not per se cause or effect the change in (Δψₘ) and Bax translocation (data not shown).

It has been known for some time that anti-apoptotic Bcl-2 family members, such as Bcl-2 and Bcl-xL, may play central roles in preventing the loss of Δψₘ, the release of cytochrome c and the initiation of apoptosis (Shimizu et al., Oncogene 13:21-29 (1996): Kim et al., Cancer Res. 57:3115-3120 (1997); Yang et al., Science 275:1129-1132 (1997)). It is also known that the level of expression of endogenous Bcl-xL, is inversely correlated with apoptotic cell death (Zhan et al., Oncogene 13:2287-2293 (1996)). The actual mechanisms responsible for the Bax conformational change, and the Bax translocation from cytosol to mitochondria remain unknown, as do the mechanism(s) responsible for the inhibition of those changes in Bax by over-expression of Bcl-2 or Bcl-xL. Nevertheless, Bax, either with Bak or alone, may play central roles in apoptosis by inducing mitochondrial dysfunction (Wei et al., 2001).

For the purposes of this invention, the terms “protein,” “peptide” and “polypeptide” are used interchangeably, the definition of which would be recognized by one in the art. An apoptotic protein, or peptide, or polypeptide refers to a compound, such as TRAIL or the TRAIL combination, which causes or accelerates apoptosis of a selected cell or cell population.

By comparison, an “apoptosis-reducing amount of apoptotic polypeptide” is meant an amount of polypeptide sufficient to prevent, inhibit, reduce, block or neutralize the percentage of cells undergoing apoptosis in a given population of cells known to undergo apoptosis, such as normal non-cancerous cells, relative to a population of untreated control cells of the same type. Preferably, the reduction in apoptosis is at least 10%, more preferably at least 50%, and even more preferably, at least 5-fold, relative to a population of untreated control cells of the same type. An “apoptosis-inhibiting amount” or “apoptosis blocking amount” would have the same meaning.

In accordance with the present invention, down regulation of apoptosis activity enhances survival of populations of cells, including the survival of cancer cells. Evaluation of the magnitude and direction of this effect indicates that reduction of the relevant caspase activity, whether accomplished upstream at the initiator stage, e.g., as in the present invention by controlling caspase activation and/or by modulating the activity of the caspase activators, or whether accomplished downstream would be equally efficacious.

“Apoptosis-associated” genes (and their polypeptide expression products) are associated with modulation, regulation or control (enhancement or reduction, respectively) of the apoptosis phenomena in a cell. By “apoptosis-regulating gene” (“apoptotic gene” or “apoptosis gene”) is meant a gene (or nucleic acid sequence or fragment or derivative thereof), which modulates, regulates or controls (either enhances or inhibits, respectively) the process of apoptosis or encodes a peptide product, such as the nucleotide sequence encoding TRAIL or the components of the TRAIL combination, that directly or indirectly regulates the process of apoptosis. By “apoptosis-regulating polypeptide” is meant a polypeptide (or protein or peptide, or active fragment, derivative, or analog thereof), such as TRAIL or the components of the TRAIL combination, which modulates, regulates or controls (either enhances or inhibits, respectively) the process of apoptosis or encodes a peptide product that directly or indirectly regulates the process of apoptosis. The term “apoptosis-regulating peptide” is used interchangeably with the terms “apoptotic polypeptide” or “apoptosis polypeptide,” as defined above.

The terms “preventing,” “blocking,” or “inhibiting,” or “reducing” or “neutralizing” and the like, are intended, using their commonly accepter meanings, to mean a reduction in cell death or a prolongation in the survival time of the cell. They also are intended to mean a diminution in the appearance or a delay in the appearance of morphological and/or biochemical changes normally associated with apoptosis. When the delay or reduction in activation or apoptotic activity is permanent, the modulating method is considered to be preventative. Thus, this invention provides compositions and methods to increase survival time and/or survival rate of a cell or population of cells which, absent the use of the method, would normally be expected to die. Accordingly, it also provides compositions and methods to prevent or treat diseases or pathological conditions associated with unwanted cell death in a population of cells or a subject.

It is important to note, when reduction, inhibition, blocking, neutralization or prevention of extrinsically-induced apoptosis is the desired modulation, as such terms are commonly used, in the present invention, that the method of this invention selectively inhibits apoptosis in normal cells, even in the presence of apoptotic-inducing agents. Accordingly, this method provides an improvement over prior art methods wherein apoptosis can be inhibited only by interfering with the induction pathway at the level of ligand induction, such as by providing antibodies or anti-ligand antibodies to interfere with the binding of the ligand to its cell surface receptor. Nevertheless, this invention can be combined with the use of such prior art methods to inhibit apoptosis.

The terms “enhancing” or “increasing” or “stimulating” or “optimizing” and the like, as such terms are commonly used, are intended to mean an increase in cell death or a reduction in the survival time of the cell. They also are intended to mean an enhanced appearance or an induced, increased or accelerated appearance of morphological and/or biochemical changes normally associated with apoptosis. Thus, this invention provides compositions and methods to decrease survival time and/or survival rate of a cell or population of cells which, absent the use of the method, would not normally be expected to die. Accordingly, it also provides compositions and methods to cause, enhance or induce apoptosis in a population of cells or a subject, e.g. in cancer cells.

“Modulation,” “regulation” or “control” are terms interchangeably intended to broadly refer to any change affecting the apoptosis of the selected cell or cell population, or the biological changes associated with apoptosis, regardless of whether the effect is inhibition or enhancement, as defined above. Thus, an “apoptosis-modulating amount” of a compound, peptide, protein, polypeptide, or the nucleic acid molecule or gene encoding same, would mean a sufficient or effective amount to enhance or reduce (depending on the desired effect) apoptosis of a cell or cell population in vitro or in vivo.

Although the preferred invention is preferably practiced in vivo using TRAIL or the TRAIL combination polypeptide, or using a specific caspase inhibitor to protect e.g., normal or selected cells from the apoptosis-inducing effect of TRAIL or the TRAIL combination, it should be apparent to those of skill in the art that the gene(s) encoding the protein expression product(s) and its biological equivalents are useful in the methods of this invention. TRAIL and its methods of isolation as well as recombinant production using the expression vectors in a host-vector system, are know and have been published in the art. Thus, methods for the production, isolation or controlled expression of the TRAIL combination would similarly be within the understanding of one of ordinary skill in the art without extensive experimentation.

Suitable cells or “target cells” for the practice of the present invention include, but are not limited to, cells in which apoptosis activity or apoptotic effect per se, or on other cells or cell populations, is modulated (inhibited or enhanced) by one or more endogenous or exogenous agents, such as TRAIL or the TRAIL combination, or under selected conditions or circumstances, e.g., mutations, chronic stress, or continuous exposure to light. In the case of normal or certain selected cells, the exogenous or extrinsic agent is a specific caspase inhibitor that protects at least a portion (preferably >50%, more preferably >75%, most preferably >90%, even more preferably >95% up to 100%) of the cells from the apoptosis-inducing effect of TRAIL or the TRAIL combination, particularly the caspase 9 inhibitor, more particularly z-LEHD-FMK.

A “target polypeptide,” refers to the protease, caspase, ligand or similar compound, which is directly or indirectly causing cell death or degeneration, or apoptosis of a cell or cell population, or which if activated (e.g., a caspase) could do so in, e.g., a population of cancer cells. To be effective in the present invention, the apoptosis-regulating or -modulating polypeptide or nucleic acid molecule expression product modulates the apoptotic or proteolytic activity of the target (either increasing or reducing the activity, as desired). Thus, it is the target polypeptide upon which the method of the present invention functions.

The cells can be mammalian cells or animal cells, such as, without limitation, guinea pig cells, rabbit cells, simian cells, mouse cells, rat cells, or human cells. The cells can be continuously cultured or isolated from an animal or human.

In one embodiment of the present invention an apoptosis-modulating amount of a protein, peptide or polypeptide, preferably TRAIL or the TRAIL combination, or active analog, fragment or derivative thereof, is administered to the patient to modulate (enhance or reduce, respectively, or both) the apoptosis of a cell or cell population in that patient. For example, ideally, TRAIL or the TRAIL combination will selectively induce apoptosis in TRAIL sensitive, and even in certain TRAIL resistant, cancer cells; while at the same time the proximate normal (non-cancerous) cells will not be affected by exposure to TRAIL or the TRAIL combination, or will be protected from apoptosis by treatment with a specific caspase inhibitor (preferably a caspase 9 inhibitor, such a z-LEHD-FMK). The preferred protein, peptide or polypeptide is administered as an isolated preparation.

As used herein, the term “substantially pure,” “an isolated preparation” or a “purified polypeptide” describes a compound, e.g., a protein or polypeptide, which has been separated from and is substantially free of components which naturally accompany it. Typically, a compound is isolated when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis or HPLC analysis.

Accordingly, this invention also provides TRAIL and a TRAIL combination polypeptide, protein, a biological equivalent thereof and fusion proteins containing these, for use in the methods described herein, including for use as a tumor-specific anti-neoplastic agent. In a preferred embodiment of the present invention, a construct was prepared in which an isoleucine-zipper (ILZ) was fused to extra-cellular portions of TRAIL and placed under the expression control of an adenovirus. Its secretion, oligomerization, and an apoptosis-inducing activity were documented in a cell culture system. Moreover, the polypeptides or proteins can be conjugated to targeting antibodies, such as the polyclonal antibody raised against TRAIL for targeted delivery.

A “biological equivalent” is intended to mean any fragment of the nucleic acid or protein, a mimetic (protein and non-protein mimetic) also having the ability to control apoptosis using the assay system described and exemplified herein. For example, purified TRAIL or the TRAIL combination polypeptide can be contacted with a suitable cell population as described above, and under such conditions apoptosis is induced in a target cell or cell population, while certain normal cells remain unaffected. Protection of normal or selected TRAIL-sensitive cells is ensured by treatment with a specific caspase inhibitor, prior to, at the time of, or very shortly after exposure of the cells to the apoptosis-inducing effect of TRAIL or the TRAIL combination.

Caspase proteases are activated in a signaling cascade, much like the blood clotting cascade, wherein each protease resides in an inactive pro-form until it is acted upon by another enzyme or protease in the cascade, which then cleaves the pro-enzyme into its active form. Once activated, that enzyme or protease then signals, acts upon or activates the next polypeptide in the signaling cascade, etc., until apoptosis is triggered in the target cell population. Therefore, if a link in the signaling cascade is blocked or sufficiently inhibited as to be ineffective in the activation of the next polypeptide in the signaling cascade, apoptosis will be reduced, inhibited or prevented. Similarly, if the signaling cascade has been blocked, but enhanced apoptosis is desired, this can be achieved by replacing the missing or ineffective polypeptide, thereby reestablishing the cascade. Moreover, only a portion of the entire primary structure may be required in order to effect biological activity. It is further understood that minor modifications of the primary amino acid sequence may result in proteins which have substantially equivalent or enhanced function as compared to the molecule within the vector. These modifications may be deliberate, as through site-directed mutagenesis, or may be accidental such as through mutation in hosts. All of these modifications are included as long as the ability to regulate apoptosis is retained.

In a preferred embodiment, the purified preparation of the isolated polypeptide having the ability to enhance or reduce apoptosis in the selected cell or cell population, preferably to modulate, regulate or control apoptosis of the target cells, is at least about 60 amino acids in length. More preferably, it is at least 120 amino acids, even more preferably, at least 200 amino acids, yet more preferably, at least 275 amino acids, and even more preferably, at least 320 amino acids in length. In an additional embodiment the polypeptide encodes the full-length protein or a regulated version thereof, e.g., provided is the TRAIL combination to induce and enhance apoptosis, even in selected TRAIL resistant cell types, and also provided is a specific caspase inhibitor (preferably a caspase 9 inhibitor, such as z-LEHD-FMX) to prevent and block apoptosis in normal cells that would otherwise be killed upon exposure to TRAIL or to the TRAIL combination.

The present invention also provides for the use of analogs of proteins or peptides capable of inducing or inhibiting apoptosis. Analogs can differ from naturally occurring proteins or peptides by conservative amino acid sequence differences or by modifications which do not affect sequence, or by both. For example, conservative amino acid changes may be made which, although they alter the primary sequence of the protein or peptide, do not normally alter its function. Conservative amino acid substitutions typically include substitutions within the following groups: glycine, alanine; valine, isoleucine, leucine; aspartic acid, glutamic acid; asparagine, glutamine; serine, threonine; lysine, arginine; phenylalanine, tyrosine. Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences, which have phosphorylated amino acid residues, e.g., phosphotyrosine, phosphoserine, or phosphothreonine.

Also included are polypeptides, which have been modified using ordinary molecular biological techniques so as to improve their resistance to proteolytic degradation or to optimize solubility properties. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

In addition to substantially full-length polypeptides, the present invention provides enzymatically active fragments of the TRAIL and TRAIL combination polypeptides, and of the caspase inhibitor protein. If the presence of the inhibitor, active fragment, derivative or homologue thereof is sufficient to block or prevent activation of apoptosis in the target cells, or to prevent expression of proteolytic or apoptotic activity in the target cells, the apoptotic modulating compound is an “apoptosis-preventing compound.” The irreversible caspase inhibitors, e.g., caspase 9 inhibitor, including z-LEHD-FMK, and caspase 2 inhibitor are examples of preferred embodiments of such an apoptosis preventing compound, although neither z-IETD-FMK (caspase 8 inhibitor) or z-VAD-FMK (Pan caspase inhibitor) are useful for the purposes of the present invention since they protect all cells, including cancer cells, from the killing effect of TRAIL.

As used herein, the term “fragment,” as applied to a polypeptide, will ordinarily be at least about 20 contiguous amino acids, typically at least about 50 contiguous amino acids, more typically at least about 70 continuous amino acids, usually at least about 100 contiguous amino acids, preferably at least about 150 continuous amino acids, more preferably at least about 200 contiguous amino acids, and most preferably at least about 250 to about 300 or more contiguous amino acids in length. An active fragment functions in much the same manner, and expresses essentially the same phenotype as the full-length expression product from which it came.

A mutant, derivative, homologue or fragment of TRAIL or the TRAIL combination, or of the caspase inhibitor protein, or of the gene(s) therefor, is also one in which selected domains in the related protein or gene share significant homology (at least about 50% homology under at least moderately stringent conditions), with the same domains in the preferred embodiment of the present invention. It will be appreciated that the definition of such an agent or compound may be applied to either amino acid or nucleic acid sequences, and encompasses those amino acid or nucleic acid molecules having at least about 40% homology of the domains contained therein, preferably at least about 50%, more preferably, at least about 60%, even more preferably, at least about 70%, even more preferably, at least about 80%, yet more preferably, at least about 90% and most preferably, the homology between similar domains is about 99%, of the polypeptides per se or of the nucleic acid encoding the expression products thereof. Homology is determinable by one of ordinary skill by simple side-by-side comparisons of the two sequences, or by any known either manual or electronic technique or method in the art.

As used herein, “nucleic acid” and “gene” are synonymous and shall mean single and double stranded genomic DNA, cDNA, mRNA and cRNA. “Isolated” when used to describe the state of the nucleic acids, denotes the nucleic acids free of at least a portion of the molecules associated with or occurring with nucleic acids in their native environment.

According to the present invention, preferably the isolated nucleic acid encoding the biologically active gene, or nucleic acid molecule, or fragment thereof, as defined above, is full-length or of sufficient length to encode an active apoptosis modulator, such as TRAIL or the TRAIL combination, or a negative regulator, such as a caspase 9 inhibitor, specifically z-LEHD-FMK. The preferred nucleic acid is at least about 200 nucleotides in length. More preferably, it is at least 400 nucleotides, even more preferably, at least 600 nucleotides, yet more preferably, at least 800 nucleotides, and even more preferably, at least about 1000 nucleotides in length.

The DNA sequence or an apoptosis-inducing or -inhibiting fragment thereof, can be duplicated using a DNA sequencer and methods well known to those of skill in the art. For example, the sequence can be chemically replicated using well known polymerase chain reaction (PCR; Perkin-Elmer) techniques, which in combination with the synthesis of oligonucleotides, allows easy reproduction of DNA sequences of up to approximately 6000 base pairs in length from as little as a single gene copy. See, e.g., U.S. Pat. Nos. 4,683,195; 4,800,159; 4,754,065, and 4,683,202.

The nucleic acid can also be duplicated using a host-vector system and traditional cloning techniques with appropriate replication vectors. A “host-vector system” refers to host cells, which have been transfected with appropriate vectors using recombinant DNA techniques. A variety of gene transfer approaches to deliver the gene into a target cell, cells or tissues include several non-viral vectors, including DNA/liposome complexes, and targeted viral protein DNA complexes. To enhance delivery to a cell, the nucleic acid or proteins of this invention can be conjugated to antibodies or binding fragments thereof which bind cell surface antigens, e.g., TCR, CD3 or CD4.

As noted above, one means to introduce the nucleic acid into the cell of interest is by the use of a recombinant expression vector. As used herein, “recombinant” is intended to mean that a particular DNA sequence is the product of various combinations of cloning, restriction, and ligation steps resulting in a construct having a sequence distinguishable from homologous sequences found in natural systems. Recombinant sequences can be assembled from cloned fragments and short oligonucleotides linkers, or from a series of oligonucleotides. “Recombinant expression vector” is intended to include vectors, which are capable of expressing DNA sequences contained therein, where such sequences are operatively linked to other sequences capable of effecting their expression. It is implied, although not always explicitly stated, that these expression vectors must be replicable in the host organisms either as episomes or as an integral part of the chromosomal DNA.

Accordingly, “expression vector” is given a functional definition, and any DNA sequence, which is capable of effecting expression of a specified DNA sequence disposed therein, is included in this term as it is applied to the specified sequence. Suitable expression vectors include viral vectors, including adenoviruses, adeno-associated viruses, retroviruses, cosmids and others. Adenoviral vectors are a particularly effective means for introducing genes into tissues in vivo because of their high level of expression and efficient transformation of cells both in vitro and in vivo, for example as disclosed in Example 6 of the present invention.

When the method of the invention is practiced in vitro, the expression vector is inserted into the target cell using well known techniques, such as transfection, electroporation or microinjection. More specifically, the in vitro method comprises providing cell cultures or tissue cultures having a cell surface receptor that mediates apoptosis, such as a TCR, the TNF receptor or the Fas receptor. The cells are cultured under conditions (temperature, growth or culture medium and gas (CO₂)) for an appropriate amount of time to attain exponential proliferation without density dependent constraints. The cells are then exposed to preliminary conditions necessary for apoptosis, for example an effective amount of an inducing agent, e.g., TRAIL or the TRAIL compound. Anti-Fas antibodies and mitogens (ConA) are well known to those of skill in the art (e.g., Itoh et al., Cell 66:233-243 (1991).

In embodiments of the invention, a virus or infective agent, or a drug or therapeutic or diagnostic agent to be tested is added in varying concentrations at a time that is simultaneous with, prior to, or after the inducing agent. The cells are assayed for caspase activity or apoptotic activity using methods well known to those of skill in the art and described herein. It is apparent to those of skill in the art that two separate cultures of cells must be treated and maintained for the test population, one as the control for the test population, while the second acts as a background control without receiving the agent to be tested.

Replication-incompetent retroviral vectors also can be used with this invention. As used herein, the terms “replication-incompetent” is defined as the inability to produce viral proteins, precluding spread of the vector in the infected host cell. The term “retroviral” includes, but is not limited to, a vector or delivery vehicle having the ability to selectively target and introduce the coding sequence into dividing cells, such as the mouse molony-leukemia virus (MMLV). As would be understood by those of skill in the art, the nucleic acid introduced with a retroviral vector would be a ribonucleic acid (RNA).

In one embodiment of this invention, the expression vector is specifically targeted to a selected cell type, such as the TRAIL-sensitive human cancer cells SW480 cells used in the Examples that follow. For these methods, it is intended that the DNA encoding TRAIL or the TRAIL combination, or the caspase inhibitor, will be operatively linked to a promoter that is highly active in the targeted cells. Expression levels of the gene or nucleotide sequence inside a target cell are capable of providing gene expression for a duration, and in an amount, such that the nucleotide product therein is capable of providing a therapeutically effective amount of gene product, or in such an amount as to provide a functional biological effect on the target cell.

By “gene delivery” is meant transportation of a composition or formulation into contact with a target cell so that the composition or formulation is capable of being taken up by means of a cytotic process (i.e., pinocytosis, endocytosis, phagocytosis, etc) into the interior or cytoplasmic side of the outermost cell membrane of the target cell where it will subsequently be transported into the nucleus of the cell in such functional condition that it is capable of achieving gene expression. By “gene expression” is meant the process, after delivery into a target cell, by which a nucleotide sequence undergoes successful transcription and translation such that detectable levels of the delivered nucleotide sequence are expressed in an amount and over a time period that a functional biological effect is achieved. “Gene therapy” encompasses the terms “gene delivery” and “gene expression.” Moreover, treatment by any gene therapy approach may be combined with other, more traditional therapies.

When the method is practiced in vivo, TRAIL or the TRAIL combination and/or a caspase inhibitor, or its expression vector can be added to a pharmaceutically acceptable carrier, and systemically administered to the subject, such as a human patient or an animal, such as a mouse, a guinea pig, a simian, a rabbit or a rat. Alternatively, it can be directly infused into the cell by microinjection. A fusion protein also can be constructed, as was done in Example 6, wherein an isoleucine-zipper (ILZ) was fused to extra-cellular portions of TRAIL and placed under the expression control of an adenovirus.

The method can also be practiced ex vivo using a modification of recognized methods. Generally, a sample of cells, such as bone marrow cells or MLC can be removed from a subject or animal using methods well known to those of skill in the art. An effective amount of the selected nucleic acid or polypeptide is added to the cells, and the cells are cultured under conditions that favor internalization of the homologous or heterologous component by the cells. The transformed cells are then returned or reintroduced to the same subject or animal (autologous) or one of the same species (allogeneic) in an effective amount and in combination with appropriate pharmaceutical compositions and carriers.

By “patient” or “subject” is meant any vertebrate or animal, preferably a mammal, most preferably a human, with apoptosis or degeneration of at least some portion of a population of cells, or with a susceptibility to, or genetic predisposition to such apoptosis or cellular degeneration in such selected population of cells. Thus, included within the present invention are animal, bird, reptile or veterinary patients or subjects, the intended meaning of which is self-evident. Despite notable differences in anatomy between the certain physical features of a primate and those of a rodent or bird, at the cellular level, cell death among other animals closely resembles that in the primate, as shown by studies made on rats, chicks and young monkeys. All are encompassed by the methods of the present invention.

In another embodiment of the present invention, the apoptosis-modulating or apoptosis-regulating compound is designed by mimetics for synthetic production. The designing of mimetics to a pharmaceutically active compound, such as the apoptosis-associated polypeptide, is a known approach to the development of pharmaceuticals based on a “lead” compound. This might be desirable where the active compound is difficult or expensive to synthesize or where it is unsuitable for a particular method of administration, e.g., peptides are unsuitable active agents for oral compositions as they tend to be quickly degraded by proteases in the alimentary canal. Similarly, the acidic environment within regions of the body, such as the vitreous of the eye, can also degrade therapeutic peptide compounds before they have had an opportunity to perform or be evaluated. Mimetic design, synthesis and testing is generally used to avoid randomly screening of a large number of molecules for a target property, for example to structurally determine which caspase inhibitors can be used to protect or rescue normal cells from apoptosis.

There are several steps commonly taken in the design of a mimetic from a compound having a given target property. Typically this can be done by systematically varying the amino acid residues in the peptide, e.g., by substituting each residue in turn. These parts or residues constituting the active region of the compound are known as its “pharmacophore.” Once the pharmacophore has been found, its structure is modeled according to its physical properties, e.g., stereochemistry, bonding, size and/or charge, using data from a range of sources, e.g., spectroscopic techniques, X-ray diffraction data and NMR. Computational analysis, similarity mapping (which models the charge and/or volume of a pharmacophore, rather than the bonding between atoms) and other techniques can be used in this modeling process.

In a variant of this approach, the three-dimensional structure of the ligand and its binding partner are modeled. This can be especially useful where the ligand and/or binding partner change conformation on binding, allowing the model to take into account the design of the mimetic. A template molecule is then selected, onto which chemical groups, which mimic the pharmacophore, are grafted. The template molecule and the chemical groups grafted on to it can conveniently be selected so that the mimetic is easy to synthesize, is likely to be pharmacologically acceptable, and does not degrade in vivo, while retaining the biological activity of the lead compound. The mimetic or mimetics found by this approach can then be screened to see whether they have the target property, or to what extent they exhibit such properties. Further optimization or modification can then be carried out to arrive at one or more final mimetics for in vivo or clinical testing, and which can then be administered to the cell population or in vivo to the cells of a patient to modulate apoptosis.

The use of the compositions and methods in vitro provides a powerful bioassay for screening for drugs that are agonists or antagonists of apoptotic functions in these cells. Thus, one can screen for drugs or agents having similar or enhanced ability to prevent or inhibit apoptosis, or in the converse to stimulate, enhance, or increase apoptosis. One of skill in the art can determine when the method has been successfully performed by noting the absence of apoptotic morphological changes or more simply, by the absence of cell death. The in vitro method further provides an assay to determine if the method of this invention is useful to treat a subject's pathological condition or disease that has been linked to, or that requires, apoptotic cell death in the individual. The percentage cell death will be determined at various times after culture by the formula: cpm released from the experimental group minus cpm of background release divided by cpm released by 0.5% Triton X-100 (complete lysis)-cpm of background released.

Accordingly, the invention further provides an assay for determining agents, which affect the expression of, or activity of, an apoptosis-associated polypeptide, such as TRAIL, the TRAIL combination or a specific caspase inhibitor, such as a caspase 9 inhibitor, preferably z-LEHD-FMX, thereby regulating or modulating apoptosis in the targeted cell or cell population, such as a population of cancer cells, or a cancer tumor. Such assay comprises administering an agent under test to the selected cell population, e.g., cancer cells at low cell density, and monitoring apoptotic death in the cells. A further assay for determining agents, which regulate or modulate an apoptosis-associated polypeptide according to the invention comprises administering the agent under test to the selected cells at high cell density, and monitoring apoptotic death in the selected cells. Agents may thus be selected which effectively may be used to regulate apoptosis. The agents which are thus selected, are also intended to be a part of the present invention.

Novel compounds or compounds identified using these assays form yet a further aspect of the invention, as does the use of known reagents identified using these assays in the modulation or control of apoptosis in a cell or cell population.

In accordance with the present invention, the apoptosis modulating polypeptides or compounds, as described above, when used in therapy, for example in the treatment of a patient, can be administered to a patient either alone or as part of a pharmaceutically acceptable composition, and optionally with a preservative, diluent, and the like. They may further be administered in the form of a composition in combination with a pharmaceutically acceptable carrier or excipient, and which may further comprise pharmaceutically acceptable salts. Examples of such “carriers,” particularly “pharmaceutically acceptable carriers,” are well known to those of skill in the art and include both liquid and solid carriers, such as water or saline, various buffer solutions, cyclodextrins and other protective carriers or complexes, emulsions, such as oil/water emulsions and various types of wetting agents, glycerol and prodrug formulations. Combinations may include other pharmaceutical agents, such as dopaminergics, adrenergics, cholinergics or growth factors.

The term “pharmaceutically acceptable salts” refers to physiologically and pharmaceutically acceptable salts of the compounds of the invention: i.e., salts that retain the desired biological activity of the parent compound and do not impart undesired toxicological effects thereto. In fact, combined therapeutics may advantageously confer selectivity in situations in which cells other than fully-differentiated cells may be encountered, e.g., cornea.

Various methods of administration of the therapeutic or preventative agent can be used, following known formulations and procedures. As used herein, the term “administering” for in vivo and ex vivo purposes means providing the subject with an effective amount of the nucleic acid molecule or polypeptide effective to prevent or inhibit apoptosis of the target cell. Administration can be effected continuously or intermittently throughout the course of treatment. Single or multiple administrations can be carried out with the dose level and pattern being selected by the treating physician. For example, the compositions can be administered at the beginning of a treatment regime to a patient already suffering from a disease or condition that is linked to apoptosis or for which programmed cell death is desired, e.g. cancer cells, without adversely affecting the surrounding tissue. In this situation, an effective “therapeutic amount” of the composition is administered to stimulate, enhance or optimize (at least partially) apoptosis in the selected cell population. In the alternative, when cell survival is desired, an effective “therapeutic amount” of the composition is administered to prevent or at least partially arrest apoptosis and the accompanying pathology, such as immunosuppression.

The pharmaceutical compositions can be administered to vertebrates, including patients, such as humans and animals (or other veterinary patients), by any method known to those of skill in the art and include, but are not limited to, microinjection, parenteral, subcutaneous, intramuscular or intravenously administration, or locally or topically by powders, ointments, salve or drops. It can also be administered orally. Similar methods of administration may be used for introducing regulatory compounds, whether the preferred embodiment introduces apoptosis-enhancing compositions or whether it introduces apoptosis-preventing compositions, depending on the need and situation.

Compounds or compositions suitable for parenteral injection comprise physiologically acceptable sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents, or vehicles include water, saline, buffered saline, dextrose, ethanol, glycerol, polyols, and the like, and suitable mixtures thereof. Proper fluidity can be maintained, for example, by the use of a coating such as lecithin, by the maintenance of the required particle size in the case of dispersions and by the use of surfactants. These compositions may also contain adjuvants, such as preserving, wetting, emulsifying, and dispensing agents.

Prevention of the action of microorganisms can be ensured by various antibacterial and antifungal agents, for example, sorbic acid, and the like. It may also be desirable to include isotonic agents, for example sugars, sodium chloride and the like. Prolonged absorption of the injectable pharmaceutical form can be brought about by the use of agents delaying absorption, for example, aluminum monostearate and gelatin.

Methods of determining the most effective means and dosage of administration are well known to those of skill in the art and will vary with the vector used for therapy, the polypeptide or protein used for therapy, the purpose of the therapy, the target cell being treated, and the subject being treated. Persons of ordinary skill can easily determine optimum dosages, dosing methodologies and repetition rates. Repetition rates for dosing can be readily estimated based upon measured residence times and concentrations of the drug in bodily fluids or tissues. The actual dosage of the modulating compound or polypeptide, such as a specific caspase inhibitor+TRAIL, preferably a caspase 9 inhibitor+TRAIL, depends on a number of factors, including the size and health of the individual patient, but generally, from 0.01 μg to 100 g per kg of body weight, inclusive are administered per day to an adult in any pharmaceutically-acceptable formulation. Optimum dosages may vary depending on the relative potency of individual polypeptides, and can generally be estimated based on EC₅₀s found to be effective in either in vitro or in vivo animal models. Doses may be given once or more daily, weekly, monthly or yearly, or even once every 2 to 20 years. Dosages of oligonucleotides or nucleic acid molecules for gene therapy will be understood by one of ordinary skill in the field of gene therapy.

Following successful treatment, it may be desirable to have the patient undergo maintenance therapy to prevent the recurrence of the disease state, wherein doses are maintained at levels ranging from 0.01 μg to 100 g per kg of body weight, once or more daily, to once every 20 years.

In a preferred embodiment of the invention, the method of treatment involves direct targeting (e.g., topical) application of the therapeutic agent to the target cell population. It may also involve the use of targeting systems, such as antibody or cell specific ligands introduced into the target cell population. Targeting may be desirable for a variety of reasons; for example if the agent is unacceptably toxic, or if it would otherwise require too high a dosage, or if it would not otherwise be able to enter the target cells.

The amount of modulating compound or polypeptide to be administered in the present method, will vary according the species of the individual, as well as the targeted cells. Instead of administering these modulating polypeptides or compounds directly, they can also be produced in the target cells by expression from an encoding gene introduced into the cells, e.g., in a viral vector. The vector could be targeted to the specific cells to be treated, or it could contain regulatory elements, which are switched on more or less selectively by the target cells.

Alternatively, the modulating polypeptide or compound of the present method is administered in a precursor form, for conversion to the active form by an activating agent produced in, or targeted to, the cells to be treated. This former type of approach involves targeting the activating or modulating polypeptide or compound to the cells by conjugation to a cell-specific antibody, while the latter involves producing the activating or modulating agent, e.g., an enzyme, in a vector by expression from encoding DNA in a viral vector.

In one embodiment, the invention provides a method of treating a patient having or at risk of having a genetic or chronic disease, wherein the disease has an etiology associated with cancer, or in the converse with an apoptotic condition, and wherein the method comprises administering to the patient a therapeutically effective amount of a composition which modulates the expression of the apoptosis gene or polypeptide in the target cell population, such that the disease is ameliorated.

By “therapeutically effective” as used herein, is meant that amount of composition that is of sufficient quantity to modulate, regulate or control the degeneration or death of the target cells. In one instance, the modulation is “amelioration,” meaning a lessening or reduction or prophylactic prevention of the detrimental effect of the disorder in the patient receiving the therapy. For example, amelioration of a cancerous condition is achieved by apoptotically causing cell death of the population of cancer cells. In another instance, the population of cells may be a population of T-cells in a patient with AIDS. In that circumstance, “amelioration” is achieved by protecting the cells from apoptosis, as one would protect normal cells when cancer cells are being treated.

It is further envisioned that under selected circumstances, controlled stimulation or enhancement of apoptosis of the target activity may be desired, in which case the term “ameliorate” will be directed to the increased or enhanced expression of the apoptosis-regulating gene or the apoptotic protein or peptide, e.g., TRAIL, the TRAIL combination, an activated caspase, or a specific caspase inhibitor, such as a caspase 9 inhibitor.

The subject of the therapeutic invention is preferably a human; however, it can be envisioned that any animal with a need for apoptosis or with an apoptotic condition of a group of cells or destruction of selected cells can be treated in the method of the present invention.

The compositions used in the methods of the present invention may additionally contain other adjunct components conventionally found in pharmaceutical compositions, at their art-established usage levels. Thus, for example, the compositions may contain additional compatible pharmaceutically-active materials such as, e.g., antipruritics, astringents, local anesthetics or anti-inflammatory agents, or may contain additional materials useful in physically formulating various dosage forms of the composition of present invention, such as dyes, flavoring agents, preservatives, antioxidants, opacifiers, thickening agents and stabilizers. However, such materials, when added, should not unduly interfere with the biological activities of the components of the compositions of the invention.

The present invention is further described in the following examples. These examples are not to be construed as limiting the scope of the appended claims. Reference is made to standard textbooks of molecular biology that contain definitions and methods and means for carrying out basic techniques, encompassed by the present invention. See, for example, Maniatis et al., Molecular Cloning: A Laboratory Manual, Cold Spring Harbor Laboratory, New York (1982) and Sambrook et al., Molecular Cloning: A Laboratory Manual, Cold Spring Harbor Laboratory, New York (1989) and the various references cited therein. These references and the previously cited publications, as well as those cited in the ensuing Examples, are expressly incorporated by reference into this specification.

### EXAMPLES

Experimental Procedures

The following experimental procedures are commonly used in the Examples provided below. Consequently, although described only once, one of ordinary skill in the art would know how to apply or combine the disclosed procedures to practice the invention as described and claimed.

Cell Lines and Culture.

Normal human primary epithelial cells (EPC2, provided by Dr. Anil Rustgi, University of Pennsylvania) and primary human foreskin keratinocytes (HFK, provided by Dr. John C. Ansel, Emory University), were grown in Keratinocyte-SFM medium (Gibco BRL, Rockville, Md.) containing bovine pituitary extract (40 μg/mL) and epidermal growth factor (1 ng/mL). The primary epithelial cells were used at passage 4 or 5. EPC2-GFP were generated by retrovirus-mediated transduction of EPC2 with a pFB-Neo retroviral vector (Stratagene, La Jolla, Calif.) containing the green fluorescent protein (GFP) cDNA. All of the human esophageal cancer cell lines (also from Dr. Rustgi's laboratory), were grown with DMEM containing 10% FCS.

TRAIL, Caspase Inhibitors, Cycloheximide, and Adriamycin.

The N-terminal histidine (His) tagged recombinant human TRAIL (rhTRAIL, Thr-95 to Gly-281) was obtained from R&D Systems (Minneapolis, Minn.). A mouse monoclonal anti-6× His antibody (R&D Systems) was used for cross-linking the rhTRAIL. The irreversible caspase inhibitors z-LEHD-FMK (caspase 9 inhibitor), z-IETD-FMK (caspase 8 inhibitor), and z-VAD-FMK (Pan caspase inhibitor) were also obtained from R&D Systems. Cells were treated with TRAIL (50 ng/mL final concentration) and 1 μg/mL of anti-6× His (N-terrninal histidine antibody) for 4 hours or for 16 hours. The caspase inhibitor was used at a final concentration of 20 μM, and was added 2 hours prior to the addition of TRAIL. When the cells were treated with both cycloheximide (CHX) and TRAIL, CHX (10 μg/mL final concentration) was added 6 hours before the addition of TRAIL. In the case of adriamycin, cells were pre-treated with adriamycin (0.5 μM final concentration) for 16 hours before the addition of TRAIL.

Assessment of TRAIL-Mediated Apoptosis.

For detecting apoptosis mediated by TRAIL, an active-caspase 3 assay was performed using Cytofix/Cytoperm kit (Pharmingen, San Diego, Calif.), as previously described (Kim et al., Int. J. Oncol. 18:241-247 (2001)). Briefly, using standard techniques, 5×10⁵ cells were seeded into a 6 well plate, after which, the cells were treated with TRAIL cross-linked with anti-6× His antibody. After 4 or 16 hours of treatment, the cells were harvested and fixed, then incubated with 0.125 μg/μl rabbit anti-active-caspase 3 Ab (Clone C92-605; Pharmingen) for 20 minutes in the dark. After washing, the cells were probed with 0.125 μg/μl of Phycoerythrin (PE)-conjugated goat anti-rabbit secondary antibody (Ab) (CALTAG Laboratories, Burlingame, Calif.) for 20 minutes in the dark. The intensity of PE was analyzed by flow cytometry using a Beckman-Coulter Epics Elite analyzer.

Western Blot Immunostaining.

Western blot analysis was carried out as previously described (Meng et al., Clin. Cancer Res. 4:251-259 (1998)). Blotted membranes were immunostained with anti-DR4 (1:500; Pharmingen), anti-DR5 (1:500; IMGENEX, San Diego, Calif.), anti-DcR1 (1:500; Pharmingen), anti-DcR2 (1:500; Pharmingen), anti-FADD (Clone IF7, 1:2000; Upstate Biotechnology, Lake Placid, N.Y.), anti-caspase 8 (Clone 5F7, 1:1000; Upstate Biotechnology), anti-FLIP (Clone Dave-2, 1:1000; Alexis Biochemicals, San Diego, Calif.), anti-Bid (1:500; Pharmingen), anti-Bax (1:500, Pharmingen), anti Bcl-2 (Bcl-2/100, 1:500; Pharmingen), anti-Bcl-xL (1:500; Pharmingen), anti-caspase 3 (E-8, 1:200; Santa Cruz, Santa Cruz, Calif.), anti-PARP (1:2000; Roche Diagnostics GmbH, Mannheim, Germany), anti-TRAIL (1:500; PeproTech Inc., Rocky Hill, N.J.), or anti-actin (I-19, 1:200; Santa Cruz).

Cell Fractionation.

To prepare cytoplasmic fraction and mitochondria-rich heavy membrane fraction, cells were harvested, washed, and then re-suspended in RSB100 (10 mM Tris-HCl, pH 7.4, 2.5 mM MgCl₂, 100 mM NaCl) containing 35 μg/mL digitonin (Calbiochem, San Diego, Calif.), and 1× complete protease inhibitor cocktail (Roche Diagnostics). After incubation on ice for 5 minutes, the cells were disrupted by passage through a 25G needle. After centrifugation at 800× g for 5 minutes at 4° C., followed by further centrifugation at 14,000× g for 10 minutes at 4° C., yielded a supernatant fraction designated by the cytoplasmic fraction. The pellet was washed once with RSB100, centrifuged once again at the above-described conditions, and re-suspended with RSB 100. This was designated as the mitochondria-rich heavy membrane fraction. Protein quantity was measured using Bio-Rad protein assay reagent (Bio-Rad, Hercules, Calif.).

Analysis of Death Inducing Signaling Complex (DISC).

To perform the DISC analysis, cells were grown, harvested and re-suspended in 2 mL of DMEM (1.5×107 cells/mL). TRAIL (500 ng/mL final concentration) and anti-6× His antibody (2.5 μg/mL final concentration) were pre-mixed for 10 minutes on ice, then added to the re-suspended cells. The cells were treated with TRAIL for 20 minutes in a 37° C. water bath with intermittent agitation. TRAIL treatment was stopped by adding 5 mL of ice-cold PBS, followed by immediate centrifugation. After washing once more with 2 mL of ice-cold PBS, the cells were lysed with IP lysis buffer (30 mM Tris-HCl, pH 7.5, 150 mM NaCl, 1% Triton-X100, 10% glycerol, and 1× complete protease inhibitor cocktail) for 30 minutes on ice. The cell lysates were cleared by centrifugation (15,000 rpm×15 minutes×2) at 4° C.

To the cleared lysate, 30 μL of protein A/G plus agarose beads (Santa Cruz) were added and incubated overnight in a cold room with continuous rocking. Then, the beads were collected by centrifugation and washed four times with IP lysis buffer. After the final wash, immunoprecipitates were eluted in 175 μL of Immunopure® Gentle Ag/Ab elution buffer (PIERCE, Rockford, Ill.) containing 0.1M DTT at room temperature for 1.5 hrs. The beads were collected again with centrifugation and the eluate (approximately 200 μL) was precipitated with a mixture of 800 μL methanol, 200 μL chloroform, and 600 μL distilled water. After centrifugation at 15,000 rpm at 4° C. for 15 minutes, the upper phase was discarded and the bottom phase was mixed with 600 μL methanol. After centrifugation at the same condition, the precipitates were re-suspended in 1× SDS loading buffer and analyzed by 12% SDS-PAGE and western immunostaining.

In the control used to verify the DISC without TRAIL stimulation, the same amounts of TRAIL and antibody were added to the lysate after the cells were lysed with IP lysis buffer. Equal amounts of total cell lysates were also analyzed as an expression control.

Detection of Mitochondrial Transmembrane Potential (Δψₘ) Change.

To detect any change of Δψₘ after treating cells with adriamycin, 3,3′-dihexyloxacarbocyanine iodide (DiOC₆ (Pan et al., 1997)) were used. Briefly, 5×10⁵ cells/well were seeded into a 6 well plate. After treatment with adriamycin, the cells were harvested, washed and re-suspended in complete media containing 100 nM DiOC₆ (Pan et al., 1997) followed by 30 minutes incubation in a 37° C. water bath. After incubation, the change in DiOC₆ (Pan et al., 1997) fluorescence was analyzed by flow cytometry using a Beckman-Coulter Epics Elite analyzer.

Suppression of Endogenous Bcl-xL Expression by Anti-Sense Bcl-xL Morpholino Oligonucleotide (AS Bcl-xL).

Anti-sense Bcl-xL morpholino oligonucleotides (5′-CGGTTGCTCTGAGACATTTTTATA-3′) (SEQID NO:2) for suppressing expression of endogenous Bcl-xL in vivo, and fluorescein-labeled standard control oligonucleotides were purchased from Gene Tools, LLC (Corvallis, Oreg.). The scrape delivery method was used to deliver the oligonucleotides to the cells. Briefly, cells (1×10⁶/well) were seeded into a 6-well culture plate, and after 16 hours of incubation, the medium was changed with 1 mL of fresh medium containing 20 μM morpholino oligonucleotides (chemically modified antisense oligonucleotides). Then, the cells were scraped out of the plates using a cell scraper (SARSTEDT, Newton, N.C.). After gently pipetting up and down twice, the cells were transferred to a new culture plate. After 48 hours of incubation, the cells were treated under the indicated conditions described in the corresponding figures for the example in which the cells were used. The addition of the morpholino groups causes cancer cells to become sensitive to TRAIL, but it does not affect cytotoxicity. The effect is not seen in normal cells.

Generation of TRAIL Expressing Adenoviruses.

To make an adenovirus expressing extra-cellular portion of TRAIL (amino acids (Aa) 95 to 281), pAdTRACK/CMV and pAdEasy systems were used as previously described (He et al., Proc. Natl. Acad. Sci. USA 95:2509-2514 (1998)). In short, the extra-cellular portion of TRAIL was amplified by PCR (polymerase chain reaction). The amplified fragment was fused directly to the signal peptide of a mouse Ig κ chain, which is secreted into the media from the cells. Then, an isoleucine zipper (ILZ) fragment was inserted between the signal peptide and TRAIL to induce self-trimerization (Harbury et al., Science 262:1401-1407 (1993)). The final resulting construct was cloned into pAdTRACK/CMV, and then a recombinant plasmid was generated in BJ5183 E. coli by co-transforming PmeI-digested TRAIL-cloned pAdTRACK/CMV and pAdEASY.

The recombinant plasmid was digested with PacI, and was used for transfection into TRAIL-sensitive human cancer 293 cells using Superfect (QIAGEN, Valencia, Calif.). Approximately five (5) days after transfection, when comet-like patches of the green cells appeared, the cells were harvested. The viruses were amplified and purified as previously described (El-Deiry et al., Cell 75:817-825 (1993)). The infectivity of the viruses was checked by the observation of green fluorescence using a fluorescence microscope. Adenoviruses expressing only GFP were also generated, and used as a control virus. When oligomer formation was checked for expressed and secreted TRAIL in the supernatant, ethylene glycol bis-(succinic acid) (EGS, final 1 mM; Sigma, St. Louis, Mo.) was included to cross-link interacting proteins, and to maintain the cross-linking during denaturing SDS-PAGE electrophoresis.

### Example 1

### TRAIL Cytotoxicity on Esophageal Cancer Cell Lines

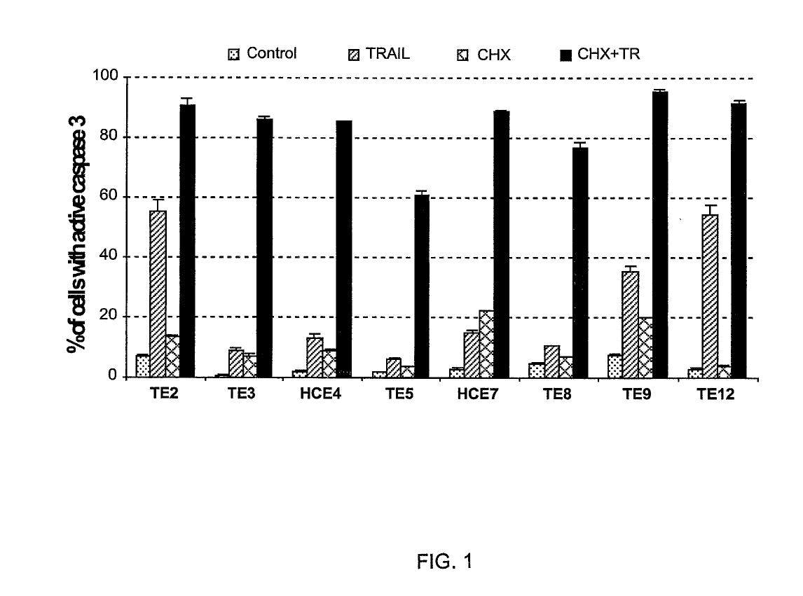

To assess TRAIL cytotoxicity on esophageal cancer cells, the active-caspase 3 assay was used. Each cancer cell population (5×10⁵ cells/well) was seeded into 6-well plates, and then the cells were treated with TRAIL cross-linked with anti-6× His antibody. After 16 hours of the TRAIL treatment, the cells were harvested and the active-caspase 3 assay was performed as described in the Experimental Procedures above. In the case of CHX treatment, the cells were pre-treated with CHX (10 μg/mL final concentration) for 6 hours before the addition of TRAIL. Using this assay, when more than 30% of the cells revealed active-caspase 3 in a cell line, the cell line was considered to be TRAIL-sensitive. Consequently, 3 esophageal cancer cell lines (TE2, TE9, and TE12) were found to be sensitive to TRAIL (FIG. 1).

### Example 2

### Relative Expression Level of Molecules in TRAIL-Mediated Apoptosis Signaling

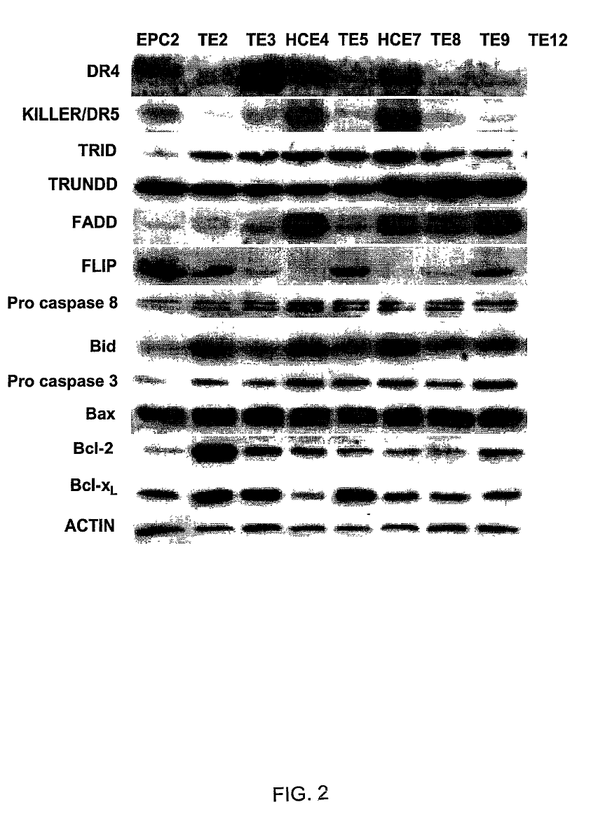

Previously, the expression level of DR4, or c-FLIP (cellular FLICE inhibitory protein; FLICE inhibits caspase 8 activation), was reported by the inventors' laboratory to be well correlated with TRAIL sensitivity (Kim et al., 2000). To confirm that correlation, and to identify possible determinants for TRAIL sensitivity in esophageal cancer cell lines, western blot immunostaining was performed (FIG. 2). Cell lysates were prepared from each cancer cell line (1×10⁶) by directly adding 1× SDS loading buffer. The samples were separated by 12% or 15% SDS-PAGE electrophoresis, and then immunostained with the antibodies shown in FIG. 2. TRAIL sensitivity was defined by the active-caspase 3 assay, and the status of p53 (Ishisaki et al., J. Biol. Chem. 273:24293-24296 (1998)) of each cell line are shown at the bottom of FIG. 2.

At the death-receptor level, pro-apoptotic death receptors were highly expressed in TRAIL-resistant cancer cells, and anti-apoptotic decoy receptors were expressed in every cancer cell line tested. Furthermore, TRID-expression in EPC2 was lower than that in the cancer cell lines.

At the DISC level, the expression level of FADD did not match with the TRAIL-sensitivity. c-FLIP, an inhibitor of caspase 8 activation, also showed no correlation with TRAIL sensitivity to the extent that it became detectable on western blot immunostaining in TRAIL-resistant HCE4 and HCE7 cells. However, it was highly expressed in TRAIL-sensitive TE2 and TE9 cells.

Caspase 8 was expressed in every cell line, and Bid was equally expressed. Moreover, there was no apparent difference in expression between cancer cell lines.

At the mitochondria level, anti-apoptotic Bcl-2 or Bcl-xL were found to be variable in expression, but TRAIL-sensitive TE2 and TE9 cells showed high-levels of expression of Bcl-2. Pro-apoptotic Bax was almost constantly expressed in the various cell lines.

Taken together, in esophageal cancers, the expression level of molecules tested in this experiment that are known to participate in TRAIL-mediated apoptosis, did not correlate well with TRAIL-sensitivity.

### Example 3

### CHX Sensitization of TRAIL-Mediated Cell Killing

The protein synthesis inhibitor, cycloheximide (“CHX”), has been widely used with apoptosis-inducing ligands, such as FAS/FASL, TNF-α, or TRAIL, to induce cell death effectively. When the cell lines were pre-treated with the protein synthesis inhibitor, CHX, all of the cancer cell lines became sensitized to TRAIL, irrespective of the sensitivity observed in response to TRAIL treatment alone (FIG. 1). c-FLIP, as an inhibitor of caspase 8 activation, has been identified as one of molecules that likely underlies TRAIL-sensitization. This is because previous observers have reported that over-expression of c-FLIP can interrupt the recruitment of caspase 8 into DISC, thereby preventing down stream activation of the caspase cascade (Wajant et al., J. Biol. Chem. 275:24357-24366 (2000)). It has also been reported that CHX might sensitize the cells to TRAIL by inhibiting a de novo synthesis of c-FLIP, thereby increasing the recruitment of caspase 8 into DISC (Fulda et al., Cancer Res. 60:3947-3956 (2000)), followed by activation of downstream apoptotic signaling cascade. However, based upon the observations of the present invention, even those cell lines that do not express c-FLIP, also became sensitized to TRAIL by CHX pre-treatment.

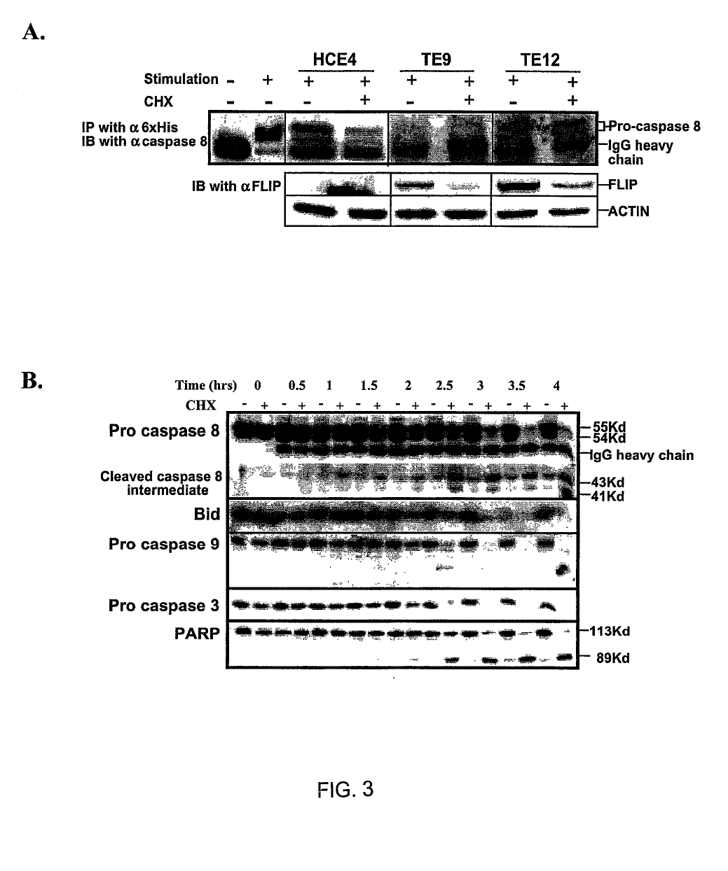

To further understand the mechanism involved in TRAIL-sensitization, tests examined whether the presence or absence of CHX pre-treatment could affect the amount of caspase 8 recruited into DISC. HCE4 and TE9 cells were used because HCE4 cells do not express c-FLIP, yet HCE4 cells are TRAIL-resistant. By contrast, TE9 cells do express c-FLIP, but TE9 cells are TRAIL-sensitive. Thus, the amount of caspase 8 recruited into DISC was compared between that in the control cells and that in the CHX-pre-treated cells. In the case of the HCE4 cells, there was a slight decrease in the amount of caspase 8 in the DISC. In the TE9 cells, despite c-FLIP becoming undetectable on western immunostaining (although the amount of caspase 8 was not affected by CHX treatment; data not shown), there was no increase in the amount of recruited caspase 8 into DISC (FIG. 3A).

FIGS. 3A and 3B depict an immunostained western blot showing the sensitization of TRAIL-resistant HCE4 cells to TRAIL resulting from CHX pre-treatment. DISC IP analysis was conducted both with or without the pre-treatment of CHX in HCE4, TE9 and TE12 cells (FIG. 3A). The DISC IP was done as described in the Experimental Procedures above, followed by western blot immunostaining to determine the recruitment of caspase 8 into DISC. Equal amounts of total cell lysates were used for western blot immunostaining to determine the expression level of c-FLIP (FIG. 3A). TRAIL cross-linked with anti-6× His antibody was added to the cells before lysis (labeled “Stimulation+” in FIG. 3A), or after lysis (labeled “Stimulation− in” FIG. 3A). Actin levels were measured as a control standard.

A time course experiment was performed using the TRAIL-resistant HCE4 cell line. After 6 hours of pre-incubation in the presence or absence of CHX, the cells were exposed to TRAIL, then lysates were prepared and harvested at time points every 30 minutes after the addition of TRAIL, up to 4 hrs by adding 1× SDS loading buffer. The timed lysates were subjected to 10 or 12% SDS-PAGE, and then immunoblotted and stained with the indicated antibodies (FIG. 3). On the resulting immunostained western blot, activation of caspase 8, Bid, caspase 9, and the cleavage of PARP (polyADP-ribose-ribose polymerase) was observed beginning approximately 1 hour after TRAIL treatment, but only in the CHX-pretreated HCE4 cells (FIG. 3B).

Taken together, these results indicate that c-FLIP does not appear to be the dominant acting inhibitor molecule in CHX-induced sensitization. There was no correlation between c-FLIP expression and TRAIL sensitivity, and the cell lines lacking expression of c-FLIP also showed the same sensitization to TRAIL after CHX pre-treatment, as the cell lines expressing c-FLIP. After CHX treatment, there was no increase in the amount of caspase 8 recruited into DISC in the esophageal cancer cell lines tested. Although c-FLIP might affect caspase 8 recruitment into DISC, other dominant inhibitor(s) that have a short half-life also apparently act at the level of caspase 8 activation. Moreover, TRAIL resistance may be governed by relative abundance of such putative inhibitor(s), rather than by any defects in the apoptotic signaling mechanisms in the tested esophageal cancer cells. This conclusion was supported by the observations that in the absence of CHX, caspase 8 was recruited without activation; whereas in the presence of CHX, there was recruitment of caspase 8 followed by activation and downstream signaling events (see FIG. 3B.) Thus, the relative expression of the putative inhibitor(s) will likely be a primary acting determinant for TRAIL-sensitivity, and regulation of its expression will lead to greater understanding of the mechanism involved in TRAIL-resistance in cancer cells.

### Example 4

### TRAIL Cytotoxicity on Normal Esophageal Epithelial Cells

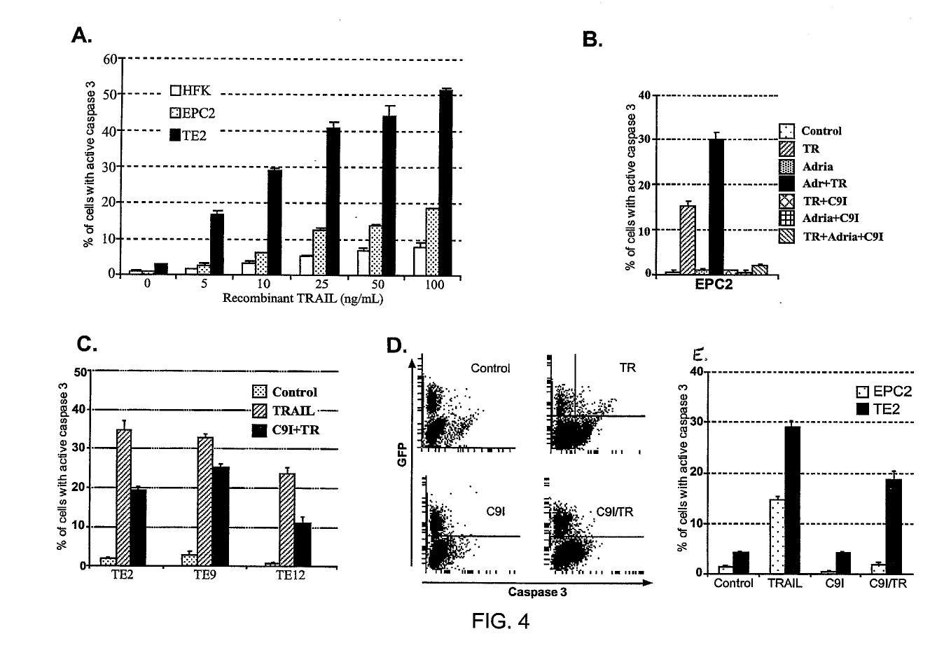

If TRAIL is cytotoxic to normal human hepatocytes, the potential use of TRAIL as a cancer chemotherapeutic drug on humans could be hampered. Recently, however, the inventors reported that z-LEHD-FMK could rescue normal human hepatocytes from TRAIL-mediated apoptosis (Ozoren et al., 2000). Accordingly, to confirm the cytoprotective effect of z-LEHD-FMK on TRAIL-mediated apoptosis in esophageal cells, HFK, EPC2, and TE2 cells were tested for TRAIL sensitivity, i.e., for apoptotic cell death (FIG. 4A). The cells were treated with increasing concentrations of TRAIL for 4 hours, then an active-caspase 3 assay was performed. Experiments were done in duplicate (mean value±SD). When compared to HFK cells, even at low concentrations of TRAIL (5, 10, 25 or 50 ng/mL), the normal esophageal epithelial EPC2 cells showed cytotoxicity in a dose dependent manner (FIG. 4A). Accordingly, the EPC2 cells were sensitive to TRAIL, while the HFK cells were not affected (FIG. 4A).

By comparison, an active-caspase 3 assay was used to determine the effect on apoptosis as a result of treatment either with TRAIL alone, or with TRAIL plus adriamycin (0.5 μM) in the presence or absence of z-LEHD-FMK on the EPC2 cells. EPC2 (5×10⁵) cells were treated with TRAIL (50 ng/mL) for 4 hours, under the conditions described above, and the results are shown in FIG. 4B. As shown, the z-LEHD-FMK completely protected the EPC2 cells from TRAIL-mediated apoptosis (FIG. 4B).

Next, to determine the effect of z-LEHD-FMK on TRAIL-sensitive esophageal cancer cells (i.e., could z-LEHD-FMK also protect TRAIL-sensitive esophageal cancer cells from apoptosis), an active-caspase 3 assay was performed, as described above in the assay shown in FIG. 4A. However, the findings showed that, even in the presence of z-LEHD-FMK, TRAIL-sensitive esophageal cancer cells still underwent apoptosis (although the extent of apoptosis was slightly reduced) (FIG. 4C).

Finally, to confirm the observation that there was preferential protection of z-LEHD-FMK on normal esophageal epithelial cells in TRAIL-mediated apoptosis, but no similar protection afforded to TRAIL-sensitive esophageal cancer cells, both EPC2-GFP (2×10⁵ cells) and TRAIL-sensitive TE2 (5×105 cells) were grown as a mixed culture in a 6-well plate. The cells were treated with TRAIL for 4 hours in the presence, or absence, of z-LEHD-FMK. An active-caspase 3 assay was performed, and the two populations within the mixed-cells were distinguished by the presence (EPC2-GFP), or absence (TE2), of GFP. The assay results showed that EPC2-GFP cells (FIG. 4D, C9I/TR) were almost completely protected from TRAIL-mediated apoptosis in the presence of z-LEHD-FMK, but the TE2 cells (FIG. 4D, C9I/TR) continued to undergo apoptosis. Experiments were done in duplicate (mean value±SD). A bar graph, prepared to display the same results quantitatively, clearly shows that the combination of the caspase 9 inhibitor and TRAIL could be used to circumvent unwanted cytotoxicity of TRAIL on normal human cells or tissues, confirming the effect previously seen in normal human hepatocytes by Ozoren et al., 2000.

### Example 5

### Synergistic Cell Killing Effect of Adriamycin and TRAIL

Previously, the inventors found that the combination of adriamycin and TRAIL could induce apoptosis synergistically in TRAIL-resistant cancer cell lines irrespective of p53 status (Kim et al, 2000). Accordingly, adriamycin was tested to determine whether its combination with TRAIL could induce synergistic cell killing in TRAIL-resistant esophageal cancer cells. TRAIL-resistant HCE4, TE3, and TE5 cells were pre-treated with 0.5 μM adriamycin for 16 hours, followed by incubation for 4 hours in the presence of TRAIL. z-LEHD-FMK (20 μM final concentration) was added 2 hours before the addition of TRAIL. An active-caspase 3 assay was used to determine whether apoptosis occurred. Indeed, it was found that when TRAIL was added to the TRAIL-resistant HCE4, TE3 and TE5 cells that had been pre-treated with adriamycin (Adria), the previously resistant cells underwent apoptosis. Moreover, the effect was synergistic as shown in FIG. 5A (Adria+TRAIL/no inhibitor).

Next, z-LEHD-FMK was tested in the HCE4, TE3 and TE5 cells to determine the protective effect of z-LEHD-FMK, if any, on the synergistic cell death driven by the combination of adriamycin with TRAIL. In case of the HCE4 cells, the cells were still killed in a synergistic way; but in the case of the TE3 and TE5 cells, the presence of z-LEHD-FMK almost completely blocked cell death (see FIG. 5A; Adria+TRAIL/C9I).

In addition to determine the potential mitochondrial membrane (Δψₘ) change after 16 hours of adriamycin (0.5 μM) pretreatment, the HCE4, TE3 and TE5 cells were incubated in the presence of 100 nM DiOC₆ (Pan et al., 1997). The change of DiOC₆ fluorescence was analyzed by flow cytometry using a Beckman-Coulter Epics Elite analyzer. However, a drop of Δψₘ was observed only in the HCE4 cells (FIG. 5B) as a result of the adriamycin treatment.

To examine the effect of the adriamycin treatment on the mitochondrial membrane potential in the TRAIL-resistant cells, cytoplasmic (cytoplasm) and mitochondrial rich heavy membrane (HM) fractions of the cell lysates were prepared, separated by SDS-PAGE electrophoresis (20 μg of cytoplasmic fraction and 5 μg of heavy membrane fraction were used per lane). The gel was immunostained, and from the resulting western blot, Bax translocation from the cytosol to the mitochondria was observed after adriamycin treatment (FIG. 5C). However, as above, the observation was made only in the HCE4 cells (FIG. 5C). Accordingly, it appeared that the discrepancy originated from the mitochondria itself. Therefore, the expression level of an anti-apoptotic Bcl-2 family member (Bcl-xL) was correlated with cell killing.

To compare the expression level of Bcl-xL, side by side, in HCE4, TE3, and TE5 cells, the same lysates used in FIG. 2 were used in a 15% SDS-PAGE. Although no correlation was observed between the Bcl-2 family and cell killing, but there was strong correlation with Bcl-xL (FIG. 5D). Thus, another cell line, TE9 was tested. TE9 cells showed only low-basal expression of Bcl-xL, and the results were the same as those from HCE4 (data not shown, and FIG. 5B and FIG. 5C).

To confirm the effect of Bcl-xL on the synergistic cell killing, we introduced anti-sense Bcl-xL morpholino oligonucleotides (AS Bcl-xL) were introduced into TE5 cells. This was to determine the effect of suppression of the endogenous Bcl-xL in the TE5 cells. Either control morpholino (“Control” or “C” in inset box FIG. 5E), or anti-sense Bcl-xL morpholino (“AS Bcl-xL” or “AS” in inset box FIG. 5E) oligonucleotides were delivered to the cells by the scrape delivery method described above. An active-caspase 3 assay was performed after each treatment, as indicated in FIG. 5E.

Upon introduction of AS Bcl-xL, the suppression of endogenous Bcl-xL was observed by western blot immunostaining (FIG. 5E). Insets in FIG. 5E show the expression level of Bcl-xL in the cells after corresponding oligonucleotide treatment. From these data it was concluded that when the expression of Bcl-xL was suppressed, the cells underwent synergistic apoptotic cell death, even in the presence of z-LEHD-FMK (FIG. 5E), in accordance with the observations in the HCE4 and TE9 cells. On the other hand, while EPC2 was also synergistically killed by the combination of TRAIL and adriamycin, it was almost completely protected from cell killing in the presence of z-LEHD-FMK (FIG. 4B).

Adriamycin did not induce drop of Δψₘ in EPC2 (data not shown), and in the normal esophageal epithelial cells the expression level of Bcl-xL is higher than that in either HCE4 or TE9 cells (FIG. 5D). Taken together, these results indicate that synergistic cell killing by the combination of adriamycin and TRIAL can be achieved in TRAIL-resistant cancer cells, but that in the presence of z-LEHD-FMK, apoptosis is not induced in normal esophageal epithelial cells. Nevertheless, the success of the combined treatment may depend on the basal expression level of anti-apoptotic Bcl-xL.

Antisense-Bcl-xL therapy directed against Bcl-xL-overexpressing tumors is indicated to be a way of sensitizing such apoptosis-resistant tumors to TRAIL-mediated therapeutic killing. Normal cells are not sensitized by antisense Bcl-xL when TRAIL is administered because the normal cells are protected by their overexpression of TRAIL decoy receptors. It is possible to combine the antisense-Bcl-xL with TRAIL plus chemotherapy, as above. As a result, the addition of antisense Bcl-xL to the present methods for treating tumors that overexpress Bcl-xL would sensitize them to TRAIL-induced apoptosis.

### Example 6

### Expression and Secretion of TRAIL and its Apoptosis-Inducing Activity

For future use for in vivo cancer therapy, adenoviruses were generated that express an isoleucine zipper (ILZ) fused with extra-cellular portions of TRAIL (“Ad-ILZ-TR;” FIG. 6A). TRAIL-resistant TE5 cells were infected with Ad-ILZ-TR (adenovirus-expressing TRAIL fused with the isoleucine zipper), and FIG. 6A presents a schematic diagram of constructs used in making Ad-GFP, and Ad-ILZ-TR. Forty-eight (48) hours after infection, western blot immunostaining was performed using lysates and supernatant to check expression and secretion (FIG. 6B). In the cell lysates, both unprocessed and processed forms of expressed TRAIL were present (FIG. 6B, lane 2), but in the supernatant, only the processed form of expressed TRAIL was present, indicating secretion of expressed TRAIL (FIG. 6B, lane 4). The same supernatant was used to confirm oligomerization of the secreted TRAIL (FIG. 6C). To maintain oligomerization of the secreted TRAIL during SDS-PAGE (12%) electrophoresis, EGS (final 1 mM) was added to the supernatant.

Next, the biological activity of the expressed TRAIL was confirmed. TRAIL-sensitive SW480 colon cancer cells were infected with Ad-GFP (FIG. 6D, a and c), or Ad-ILZ-TR (FIG. 6D, b and d). The cells infected with Ad-ILZ-TR were morphologically apoptotic (FIG. 6D, b and d).

Western blot immunostaining, performed using lysates prepared from SW480 cells infected with Ad-GFP, or Ad-ILZ-TR (matched to the evaluation in FIG. 6D), revealed that caspase 3 and PARP were cleaved in the cells infected with Ad-ILZ-TR (FIG. 6E, lane 2). However, when z-VAD-FMK (20 μM final) was added at a time point 1 hour after the onset of virus infection, no cleavage was observed (FIG. 6E, lane 4) in the SW480 cells, and the cells were protected.

When the SW480 cells were treated with supernatant from the TE5 cells infected with each corresponding virus, the effect was the same as infection (data not shown). Then, the effect of the viruses (Ad-ILZ-TR derived TRAIL) was verified on TRAIL-sensitive TE2 cells, TRAIL-resistant TE5 cells, and normal esophageal epithelial EPC2 cells. As expected, based upon the earlier observations, TE2 cells were apoptotic (FIG. 6F, TE2), but TE5 cells were resistant to infection (FIG. 6F, TE5). EPC2 cells were undergoing apoptosis (FIG. 6F; b and e; lanes 2 and 8), but in the presence of z-LEHD-FMK, the normal cells were completely protected from cell death (FIG. 6F; c and f; lanes 3, and 9).

In sum, although TRAIL (or Apo2L) is a potent chemotherapeutic reagent for inducing cell death of diverse tissue types of cancers, but not in normal cells, certain cancer cells remained resistant to TRAIL-induced apoptosis. The present invention, however, provides evidence that treatment of the TRAIL-resistant cancer cells with TRAIL in combination with a reagent that acts on the mitochondrial pathway, such as adriamycin, rapidly and synergistically induced cell death in the remaining cells, which unfortunately included the normal cells. Nevertheless, proper treatment of the cells with a caspase 9 inhibitor, such as z-LEHD-FMK, completely protected the normal cells from TRAIL-mediated apoptosis, either by exposure to TRAIL alone, or by the TRAIL combination. Meanwhile, with the exception of certain high Bcl-xL expressing cancer cells, the otherwise TRAIL-resistant cancer cells were killed, even in the presence of the caspase 9 inhibitor. Additionally, constructs were generated comprising adenovirus-controlled expression of an isoleucine-zipper fused to extra-cellular portions of TRAIL, and its secretion, oligomerization, and an apoptosis-inducing activity were documented in cell culture systems. Taken together, the present invention comprises methods, wherein the proper use of a caspase specific inhibitor, such as a caspase 9 inhibitor, makes the use of systemic (or local) TRAIL (alone or in combination with a reagent that acts on the mitochondrial pathway) safer for use in the presence of normal cells, while at the same time providing a significantly more powerful cancer therapy for humans.

Each and every patent, patent application and publication that is cited in the foregoing specification is herein incorporated by reference in its entirety.

While the foregoing specification has been described with regard to certain preferred embodiments, and many details have been set forth for the purpose of illustration, it will be apparent to those skilled in the art without departing from the spirit and scope of the invention, that the invention may be subject to various modifications and additional embodiments, and that certain of the details described herein can be varied considerably without departing from the basic principles of the invention. Such modifications and additional embodiments are also intended to fall within the scope of the appended claims.

We claim:

1. A method of controlling, modulating or regulating extrinsically-induced apoptosis in a population of cells comprising treating the cell population with a synergistically combined composition, comprising an amount of TRAIL in conjunction with an amount of at least one reagent acting on mitochondrial pathways of the cells, which combination is sufficient to induce cellular apoptosis, and wherein the apoptotic effect of TRAIL alone or the at least one reagent alone, or the additive individual apoptotic effects of TRAIL and the at least one reagent, fail to achieve the apoptotic effect on the cell population of the synergistic combination.

2. The method of claim 1, wherein treating with the synergistic combination further comprises exposing the cell population first to TRAIL, followed by the at least one reagent acting on the mitochondrial pathway.

3. The method of claim 2, wherein the population of cells being treated comprises cancer cells.

4. The method of claim 3, wherein the cancer cells being treated comprise TRAIL resistant cancer cells.

5. The method of claim 4, wherein treating the population of cells with the synergistic combination further comprises causing at least a portion of the TRAIL resistant cancer cells to become sensitive to apoptosis extrinsically induced by the synergistic combination.

6. The method of claim 3, wherein the population of cells further comprises normal, non-cancerous cells, sensitive to apoptosis extrinsically induced by the synergistic combination, and wherein the method further comprises preventing, inhibiting, or blocking the apoptosis extrinsically induced by the synergistic combination, and effecting resistance to the synergistic combination in the normal cells by treating the cell population with an amount of a specific caspase inhibitor sufficient to induce resistance to the synergistic combination.

7. The method of claim 6, wherein the specific caspase inhibitor is a caspase 9 inhibitor.

8. The method of claim 7, wherein the caspase 9 inhibitor is z-LEHD-FMX.

9. The method of claim 6, further comprising rescuing a portion of the normal cells, wherein that portion are undergoing apoptosis extrinsically induced by the synergistic combination, stopping the extrinsically induced apoptosis and effecting resistance to the synergistic combination in at least that portion of the cells by treating the cell population with an amount of the specific caspase inhibitor sufficient to induce resistance to the synergistic combination.

10. The method of claim 9, wherein the specific caspase inhibitor is a caspase 9 inhibitor.

11. The method of claim 11, wherein the caspase 9 inhibitor is z-LEHD-FMX.

12. The method of claim 1, wherein the at least one reagent acting on mitochondrial pathways of the cells is a chemotherapeutic composition.

13. The method of claim 12, wherein the chemotherapeutic composition is selected from the group consisting of doxorubicin (adriamycin), cisplatin, carboplatin, etoposide, cyclophosphamide, 5FU, CPT11 (irinotecan), taxol or derivatives thereof, vincrisitine and vinerolbine.

14. The method of claim 1, wherein the at least one reagent acting on mitochondrial pathways of the cells results in vivo from a chemotherapeutic treatment.

15. The method of claim 14, wherein the at least one reagent is produced as a result of chemotherapeutic radiation.

16. A method for preventing, inhibiting, or blocking extrinsically TRAIL-induced apoptosis in a population of normal, non-cancerous, TRAIL-sensitive cells exposed to TRAIL, comprising treating the cell population with an amount of a specific caspase inhibitor sufficient to induce TRAIL resistance in the normal cells.

17. The method of claim 16, wherein the specific caspase inhibitor is a caspase 9 inhibitor.

18. The method of claim 17, wherein the caspase 9 inhibitor is z-LEHD-FMX.

19. The method of claim 16, further comprising rescuing a portion of the normal cells, wherein that portion are undergoing TRAIL-induced apoptosis, stopping the extrinsically induced apoptosis and effecting TRAIL resistance in at least that portion of the cells by treating the cell population with an amount of the specific caspase inhibitor sufficient to induce resistance to TRAIL.

20. The method of claim 19, wherein the specific caspase inhibitor is a caspase 9 inhibitor.

21. The method of claim 20, wherein the caspase 9 inhibitor is z-LEHD-FMX.

22. A composition of matter comprising TRAIL in conjunction with at least one reagent acting on cellular mitochondrial pathways, which in combination synergistically achieves extrinsically-induced apoptosis in a population of cancer cells greater than the effect of TRAIL alone or the effect of the at least one reagent alone, or of the additive combined individual effects of TRAIL and the at least one reagent.

23. The composition of claim 22, wherein the synergistic combination is prepared in vitro, or the combined effect is achieved in vivo by the administration of TRAIL and the at least one reagent acting on the mitochondrial pathway.

24. The composition of claim 23, wherein the at least one reagent acting on mitochondrial pathways of the cells is a chemotherapeutic composition.

25. The method of claim 24, wherein the chemotherapeutic composition is selected from the group consisting of doxorubicin (adriamycin), cisplatin, carboplatin, etoposide, cyclophosphamide, 5FU, CPT11 (irinotecan), taxol or derivatives thereof, vincrisitine and vinerolbine.

26. The method of claim 23, wherein the at least one reagent acting on mitochondrial pathways of the cells results in vivo from a chemotherapeutic treatment.

27. The method of claim 26, wherein the at least one reagent is produced as a result of chemotherapeutic radiation.

28. A therapeutic method of treating a cancer patient by administering to the patient having a population of cancer cells an apoptosis-inducing amount of a synergistically combined composition comprising an amount of TRAIL in conjunction with an amount of at least one reagent acting on mitochondrial pathways of cancer cells, which in combination is sufficient to induce cellular apoptosis, wherein the effect of TRAIL alone or the at least one reagent alone, or the additive individual effects of TRAIL and the at least one reagent, fail to achieve the cellular apoptosis effect of the synergistic combination.

29. The therapeutic method of claim 28, wherein the cellular apoptotic effect of the administered synergistic combination is systemic.

30. The therapeutic method of claim 28, wherein the cellular apoptotic effect of the administered synergistic combination is localized to a selected population of cells.

31. The therapeutic of claim 28, wherein the population of cancer cells comprises, at least in part, TRAIL-resistant cancer cells, and wherein the method further comprises causing at least a portion of the TRAIL-resistant cancer cells to become sensitive to apoptosis extrinsically-induced by the synergistic combination.

32. The method of claim 28, wherein the population of cells further comprises normal, non-cancerous cells, sensitive to apoptosis extrinsically induced by the synergistic combination, and wherein the method further comprises preventing, inhibiting, or blocking the apoptosis extrinsically induced by the synergistic combination or rescuing cells undergoing apoptosis, and effecting resistance to the synergistic combination in the normal cells by treating the cell population with an amount of a specific caspase inhibitor sufficient to induce resistance to the synergistic combination.

33. The method of claim 32, wherein the specific caspase inhibitor is a caspase 9 inhibitor.

34. The method of claim 33, wherein the caspase 9 inhibitor is z-LEHD-FMX.

35. The method of claim 28, wherein the at least one reagent acting on mitochondrial pathways of the cells is a chemotherapeutic composition.

36. The method of claim 35, wherein the chemotherapeutic composition is selected from the group consisting of doxorubicin (adriamycin), cisplatin, carboplatin, etoposide, cyclophosphamide, 5FU, CPT11 (irinotecan), taxol or derivatives thereof, vincrisitine and vinerolbine.

37. The method of claim 28, wherein the at least one reagent acting on mitochondrial pathways of the cells is produced in vivo as a result of a chemotherapeutic treatment.

38. The method of claim 37, wherein the at least one reagent is produced as a result of chemotherapeutic radiation.

39. A construct comprising an adenovirus-controlled expression of an isoleucine-zipper fused to at least one extra-cellular portion of TRAIL.

40. The method of claim 1, further comprising, when the cells are apoptosis-resistant, Bcl-xL overexpressing cells, pretreating the cells with antisense Bcl-xL therapy to sensitize the apoptosis-resistant tumors to TRAIL-mediated therapeutic killing, followed by treating the cells with the synergistic combination of TRAIL in conjunction with an amount of at least one reagent acting on mitochondrial pathways of the cells, which combination is sufficient to induce cellular apoptosis in the antisense Bcl-xL sensitized cells.


SEQUENCE LISTING (2 sequences)

SEQ ID NO: 1 - protein, 4 aa, Artificial Sequence
  Description of Artificial Sequence commercially available
LEHD

SEQ ID NO: 2 - DNA, 24 bp, Unknown Organism
  Description of Unknown Organismanti-sense Bcl-xl morpholino oligonucleotide
cggttgctctgagacatttttata